(19) United States
(12) Patent Application Publication
(10) Pub. No.: US 2023/0330278 A1
(43) Pub. Date: Oct. 19, 2023

(54) METHODS FOR RADIOLABELLING PSMA BINDING LIGANDS AND THEIR KITS

(71) Applicant: Novartis AG, Basel (CH)

(72) Inventors: Donato BARBATO, Ivrea (IT); Elena CASTALDI, Ivrea (IT); Lorenza FUGAZZA, Ivrea (IT); Mattia TEDESCO, Alba (Cuneo) (IT)

(21) Appl. No.: 17/997,295

(22) PCT Filed: Apr. 28, 2021
(86) PCT No.: PCT/EP2021/061137

§ 371 Date: Oct. 27, 2022

(30) Foreign Application Priority Data
Apr. 29, 2020 (EP) 20172119.8

(51) Int. Cl. A61K 51/04; A61K 51/12; A61K 51/08

(52) U.S. Cl. CPC A61K 51/0497; A61K 51/121; A61K 51/088; A61K 51/0402

(57) ABSTRACT

The present disclosure relates to methods for radiolabelling PSMA binding ligands with a radioactive isotope, preferably ⁶⁸Ga, ⁶⁷Ga or ⁶⁴Cu, and their kits.

### TECHNICAL FIELD

The present disclosure relates to methods for radiolabelling PSMA binding ligands, and their kits.

### BACKGROUND

Prostate cancer is one of the most widespread cancers in the US and in Europe. In particular, metastatic prostate cancer (mCRPC) is associated with poor prognosis and diminished quality of life.

Recently, a new development stream for treating prostate cancer is represented by the endo-radiotherapy based on PSMA ligands, as PSMA is considered to be a suitable target for imaging and therapy due to its over-expression in primary cancer lesions and in soft-tissue/bone metastatic disease. Also, PSMA expression seems to be even higher in the most aggressive castration-resistant variants of the disease, which represents a patient population with high unmet medical need. (Marchal et al., Histol Histopathol, 2004, July; 19(3):715-8; Mease et al., Curr Top Med Chem, 2013, 13(8):951-62).

Among many small-molecule ligands targeting PSMA, the urea-based low molecular weight agents have been the most extensively investigated ones. These agents were shown to be suitable for prostate cancer clinical assessment as well as for PRRT therapy (Kiess et al., Q J Nucl Med Mol Imaging, 2015; 59:241-68). Some of these agents have glutamate-urea-lysine (GUL) as the targeting scaffold. A class of molecules was created following the strategy to attach a linker between the chelator and GUL moiety. This approach allows the urea to reach the binding site while keeping the metal chelated portion on the exterior of the binding site. This strategy was successful in xenograft PSMA positive tumors due to its demonstrated high uptake and retention as well as fast renal clearance (Banerjee et al., J Med Chem, 2013; 56:6108-21). It has also been shown that this class of molecule can be labeled with ⁶⁸Ga, and used it in the detection of prostate cancer lesions by PET imaging (Eder et al. Pharmaceuticals 2014, 7, 779-796).

However, no optimized method has been developed for labeling PSMA binding ligand with ⁶⁸Ga, ⁶⁷Ga or ⁶⁴Cu to thereby obtain labeled PSMA binding ligand solution for imaging purposes of prostate cancer tumors in human patients. In particular, there is need for a rapid, efficient and safe procedure which would provide a high radiochemical purity of labeled PSMA binding ligand, such as [⁶⁸Ga] PSMA binding ligand for intravenous injection in human subject in need thereof.

### SUMMARY

One first aspect of the disclosure relates to a method for labeling a PSMA binding ligand with a radioactive isotope, preferably ⁶⁸Ga, ⁶⁷Ga or ⁶⁴Cu, said method comprising the steps of:
i. providing a first vial comprising said PSMA binding ligand, and optionally a bulking agent, in dried form,
ii. adding a solution of said radioactive isotope into said first vial, thereby obtaining a solution of said PSMA binding ligand with said radioactive isotope,
iii. mixing the solution obtained in ii. with at least a buffering agent, and incubating it for a sufficient period of time for obtaining said PSMA binding ligand labeled with said radioactive isotope, and,
iv. optionally, adjusting the pH of the solution.

In specific embodiments, said radioactive isotope is ⁶⁸Ga and the radiochemical purity as measured in HPLC is at least 92%, and optionally, the percentage of free ⁶⁸Ga³⁺ (in HPLC) is 2% or less, and/or the percentage of non-complexed ⁶⁸Ga³⁺ species (in ITLC) is 3% or less.

In other specific embodiments, said radioactive isotope is ⁶⁷Ga and the radiochemical purity as measured in HPLC is at least 90%, and optionally, the percentage of free ⁶⁷Ga³⁺ (in HPLC) is 2% or less, and/or the percentage of non-complexed ⁶⁷Ga³⁺ species (in ITLC) is 5% or less.

In other specific embodiments, said radioactive isotope is ⁶⁴Cu and the radiochemical purity as measured in HPLC is at least 92%, and optionally, the percentage of free ⁶⁴Cu²⁺ (in HPLC) is 2% or less, and/or the percentage of non-complexed ⁶⁴Cu²⁺ species (in ITLC) is 3% or less.

Preferably, the PSMA binding ligand is a compound of formula (I):

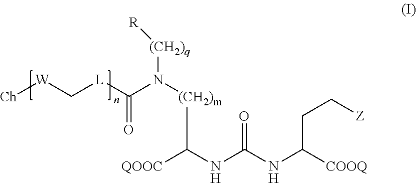

wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to ring atoms, said aryl and heteroaryl being substituted 1 or more times with X;
X is -V-Y;
V is a bond or a C₁-C₆ alkylene, preferably V is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′R′″)═NR″″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR₂—(C═O), —NR₂—(C═S), —(C═O)—NR₂—, and —(C═S)—NR₂—, preferably, W is —(C═O)—NR₂—;
each occurrence of L and W can be the same or different;
R² is H or C₁-C₄ alkyl, preferably R² is H;
n is an integer selected from the group consisting of 1, 2 and 3;
Ch is a chelating agent, typically DOTA.

In another aspect, the disclosure relates to a solution comprising a PSMA binding ligand labeled with a radioactive isotope, obtainable or obtained by the method, for use as an injectable solution for in vivo detection of tumors, typically PSMA-expressing tumors, by imaging in a subject in need thereof.

It is another object of the present disclosure to provide a powder for solution for injection, comprising the following components in dried forms:
i. a PSMA binding ligand of formula (I):

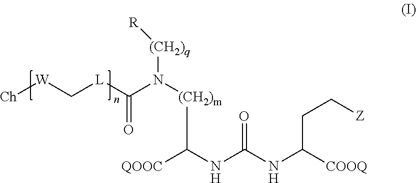

wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is -V-Y;
V is a bond or a C₁-C₆ alkylene, preferably V is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′″)═NR″″, —S(O)R′, —S(O)₂R′, —S(O)₂NR′R″, —NRSO₂R′, —CN and —NO₂, in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR₂—(C═O), —NR₂—(C═S), —(C═O)—NR₂—, and —(C═S)—NR₂—, preferably, W is —(C═O)—NR₂—;
each occurrence of L and W can be the same or different;
R² is H or C₁-C₄ alkyl, preferably R² is H;
n is an integer selected from the group consisting of 1, 2 and 3;
Ch is a chelating agent, typically DOTA; and
   ii. a bulking agent, for example, mannitol.

Typically, said powder for solution for injection comprises the following components:
i. a PSMA binding ligand of formula (II) in an amount between 10 and 100 μg, preferably between 15 and 60 μg, even more preferably about 30 μg;

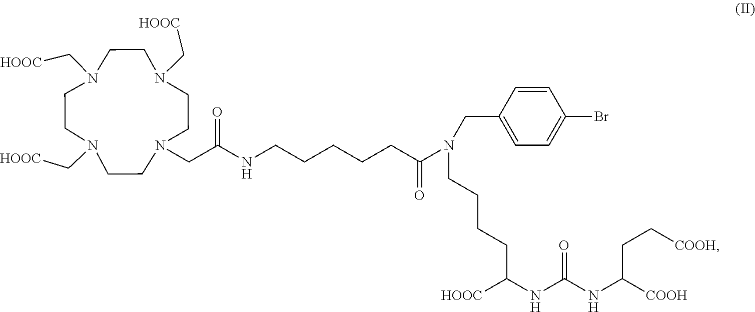 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. mannitol in an amount between 5 and 50 mg, preferably between 10 and 30 mg, even more preferably about 20 mg.

The present disclosure further relates to a kit for carrying out the method, comprising
i. a first vial with the following components in dried forms
   i. a PSMA binding ligand of formula (II):

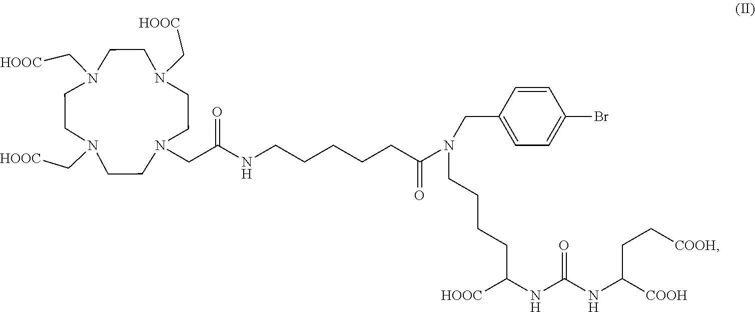 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. optionally a bulking agent, for example, mannitol, and,
ii. a second vial comprising at least a buffering agent, preferably in dried form; and,
iii. optionally, an accessory cartridge for eluting a radioactive isotope generated by a radioactive isotope generator or a cyclotron.

Another kit herein disclosed comprises:
i. a single vial with the following components, preferably in dried forms:
   i. a PSMA binding ligand of formula (II):

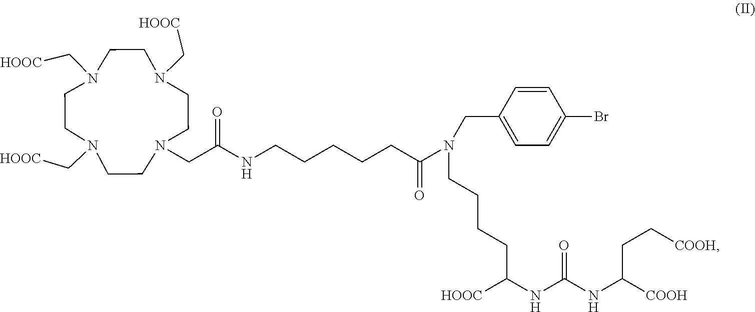 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. optionally a bulking agent, for example, mannitol,
iii. at least a buffering agent, and,
ii. optionally, an accessory cartridge for eluting a radioactive isotope generated by a radioactive isotope generator or a cyclotron.

For example, the kit may comprise a first or single vial with the following components:
i. a PSMA binding ligand of formula (II) in an amount between 10 and 100 μg, preferably between 15 and 60 μg, even more preferably about 30 μg

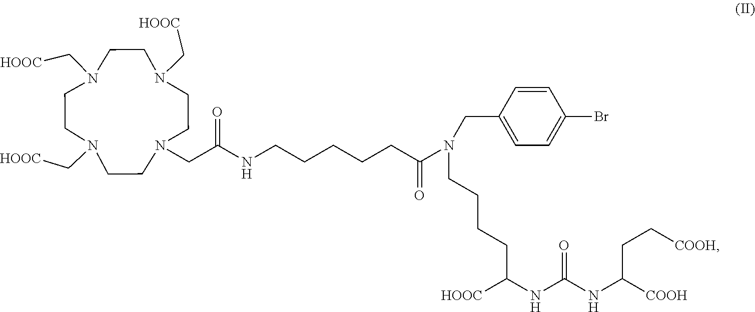 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. mannitol in an amount between 5 and 50 mg, preferably between 10 and 30 mg, even more preferably about 20 mg .

### DETAILED DESCRIPTION OF THE INVENTION

In general, the present disclosure relates to a method for labeling a PSMA binding ligand with a radioactive isotope, preferably ⁶⁸Ga, ⁶⁷Ga or ⁶⁴Cu, said method comprising the steps of:
i. providing a first vial comprising said PSMA binding ligand, and optionally a bulking agent, in dried form,
ii. adding a solution of said radioactive isotope into said first vial, thereby obtaining a solution of said PSMA binding ligand with said radioactive isotope,
iii. mixing the solution obtained in ii. with at least a buffering agent, and incubating it for a sufficient period of time for obtaining said PSMA binding ligand labeled with said radioactive isotope, and,
iv. optionally, adjusting the pH of the solution.

The radiolabeled PSMA binding ligand obtained by the disclosed methods is preferably a radioactive PSMA binding ligand for use as a contrast agent for PET/CT, SPECT or PET/MRI imaging. In a preferred embodiment, ⁶⁷Ga is used for SPECT imaging and ⁶⁸Ga and ⁶⁴Cu are used for PET imaging such as PET/CT or PET/MRI

A preferred radiolabeled PSMA binding ligand obtained by the disclosed methods is the PSMA binding ligand of formula (II):

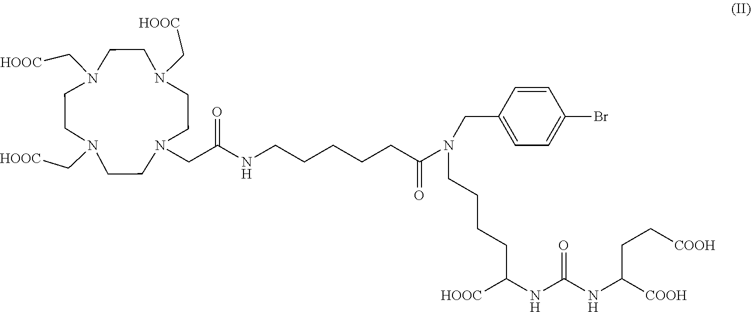 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

labelled with a radioactive isotope suitable for use as a contrast agent for PET/CT, SPECT or PET/MRI imaging, preferably ⁶⁸Ga, ⁶⁷Ga or ⁶⁴Cu.

The methods of the present disclosure may advantageously provide excellent radiochemical purity of the radiolabelled compound, e.g. radiolabeled PSMA binding ligand of formula (II) with ⁶⁸Ga, typically the radiochemical purity as measured in HPLC is at least 92%, and optionally, the percentage of free ⁶⁸Ga³⁺ (in HPLC) is 2% or less, and/or the percentage of non-complexed ⁶⁸Ga³⁺ species (in ITLC) is 3% or less.

Assays for measuring radiochemical purity in HPLC or in ITLC and free ⁶⁸Ga³⁺ are further described in detail in the Examples.

### Definitions

The terms “PSMA binding ligand” and “PSMA ligand” are used interchangeably in the present disclosure. They refer to a molecule capable of interacting, for example binding, with the PSMA enzyme.

The phrase “treatment of” and “treating” includes the amelioration or cessation of a disease, disorder, or a symptom thereof. In particular, with reference to the treatment of a tumor, the term “treatment” may refer to the inhibition of the growth of the tumor, or the reduction of the size of the tumor.

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, “MRI” stands for magnetic resonance imaging.

As used herein, “CT” stands for computed tomography.

As used herein, the terms “effective amount” or “therapeutically efficient amount” of a compound refer to an amount of the compound that will elicit the biological or medical response of a subject, for example, ameliorate the symptoms, alleviate conditions, slow or delay disease progression, or prevent a disease.

As used herein, the terms “substituted” or “optionally substituted” refers to a group which is optionally substituted with one or more substituents selected from: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —(O)₂NR′R″, —NRSO₂R′, —CN, —NO₂, —R′, —N₂—CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, alkyl, heteroalkyl, cycloalkyl, heterocycloalkyl, aryl and heteroaryl. When a compound of the disclosure includes more than one R group, for example, each of the R groups is independently selected as are each R′, R″, R′″ and R″″ groups when more than one of these groups is present.

As used herein, the terms “alkyl”, by itself or as part of another substituent, refer to a linear or branched alkyl functional group having 1 to 12 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

As used herein, the terms “heteroaryl” refer to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “aryl” refer to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto. Suitable aryl groups include phenyl, naphtyl and phenyl ring fused to a heterocyclyl, like benzopyranyl, benzodioxolyl, benzodioxanyl and the like.

As used herein, the term “halogen” refers to a fluoro (—F), chloro (—Cl), bromo (—Br), or iodo (—I) group

As used herein, the term “in dried form” refers to a pharmaceutical composition that has been dried to a powder having a moisture content below about 10% by weight, usually below about 5% by weight, and preferably below about 3%.

As used herein, the term “chelator” refers to a molecule with functional groups such as amines or carboxylic group suitable to complex the radioactive isotope via non-covalent bonds.

As used herein, the term “antioxidant” refers to a compound that inhibits oxidation of organic molecules. Antioxidants include gentisic acid and ascorbic acid.

As used herein, the term “Radiochemical purity” refers to that percentage of the stated radionuclide that is present in the stated chemical or biological form. Radiochromatography methods, such as HPLC method or instant Thin Layer Chromatography method (iTLC), are the most commonly accepted methods for determining radiochemical purity in the nuclear pharmacy.

### Step (i) of Providing a First Vial Comprising said PSMA Binding Ligand in Dried Form

The PSMA Binding Ligand

Advantageously, the PSMA binding ligand is a molecule comprising a) a urea of 2 amino- acid residues, typically a glutamate-urea-lysine (GUL) moiety, and b) a chelating agent which can coordinate radioactive isotope.

According to an embodiment, the PSMA binding ligand is a compound of formula (I):

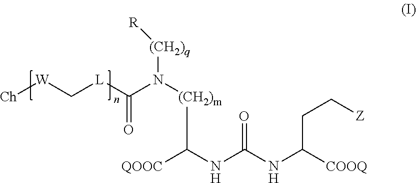

wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to ring atoms, said aryl and heteroaryl being substituted 1 or more times with X;
X is -V-Y;
V is a bond or a C₁-C₆ alkylene, preferably V is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′″)═NR″″, —S(O)R′, —S(O)₂R′, —S(O)₂NR′R″, —NRSO₂R′, —CN and —NO₂ in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR²—(C═O), —NR₂—(C═S), —(C═O)—NR₂—, and —(C═S)—NR²—, preferably, W is —(C═O)—NR₂—;
each occurrence of L and W can be the same or different;
R² is H or C₁-C₄ alkyl, preferably R² is H;
n is an integer selected from the group consisting of 1, 2 and 3;
Ch is a chelating agent, typically DOTA.

Compounds of formula (I) include the stereoisomers of formulae (Ia), (Ib), (Ic) and (Id):

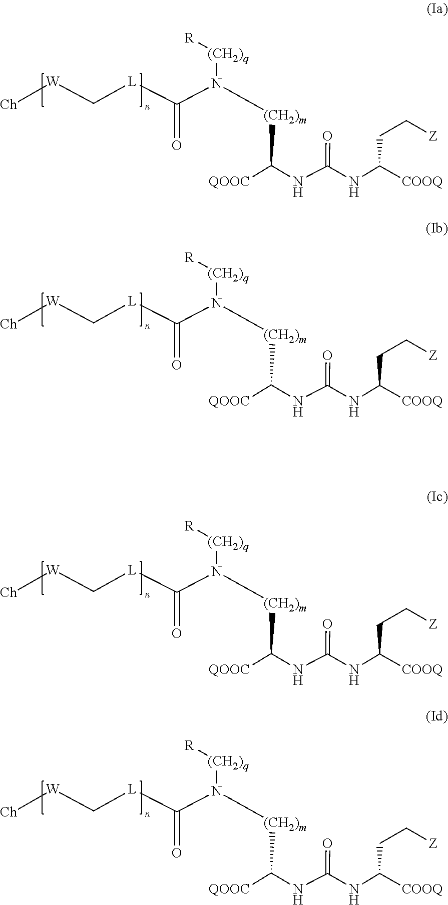

The phrase “wherein each occurrence of L and W can be the same or different” means that when the variable “n” is 2 or 3, one “L” group can be C₁-C₆ alkylene, whereas the other “L” group or groups can be C₃-C₆ cycloalkylene or arylene, or, in other embodiments, each “L” group can be, for example, C₁-C₆ alkylene. Likewise, for example, when “n” is 2 or 3, one “W” group can be —(C═O)—NR₂— and the other “W” group or groups can be —(C═S)—NR₂—, or, in other embodiments, each “W” can be, for example, —(C═O)—NR₂—.

According to an embodiment, L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, —NR′R″, -halogen, —OC(O)R′, —C(O)R′, —CO2R′, —C(O)NR′R″, —OC(O)NR′R″, —NR″C(O)R′, —NR′—C(O)NR″R′″, —NR″C(O)OR′, in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl.

According to an embodiment, L is a linker selected from the group consisting of C₃-C₆ alkylene optionally substituted with one or more substituents selected from: —OR′, ═O, ═NR′, —NR′R″, -halogen, —OC(O)R′, —C(O)R′, —CO2R′, —C(O)NR′R″, —OC(O)NR′R″, —NR″C(P)R′, —NR′—C(O)NR″R′″, —NR″C(O)OR′, in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl.

According to an embodiment, R is selected from the group consisting of C₆-C₁₀ aryl substituted with one or more halogen and pyridine substituted with one or more halogen.

According to an embodiment, R is selected from the group consisting of:

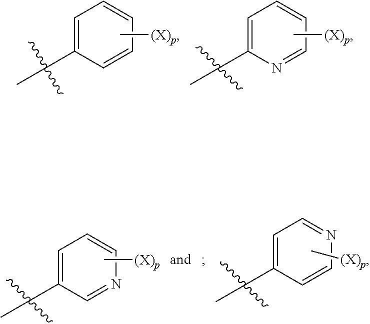

wherein p is an integer selected from the group consisting of 1, 2, 3, 4, and 5, preferably p is 1.

According to a specific embodiment, R is selected from

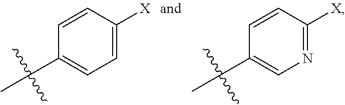

and, more preferably R is

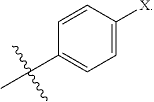

According to a specific embodiment, X is selected from Br and I.

Advantageously, R is

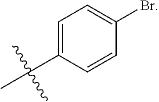 [SMILES: *c1ccc(Br)cc1]

Ch can be selected from the group consisting of:

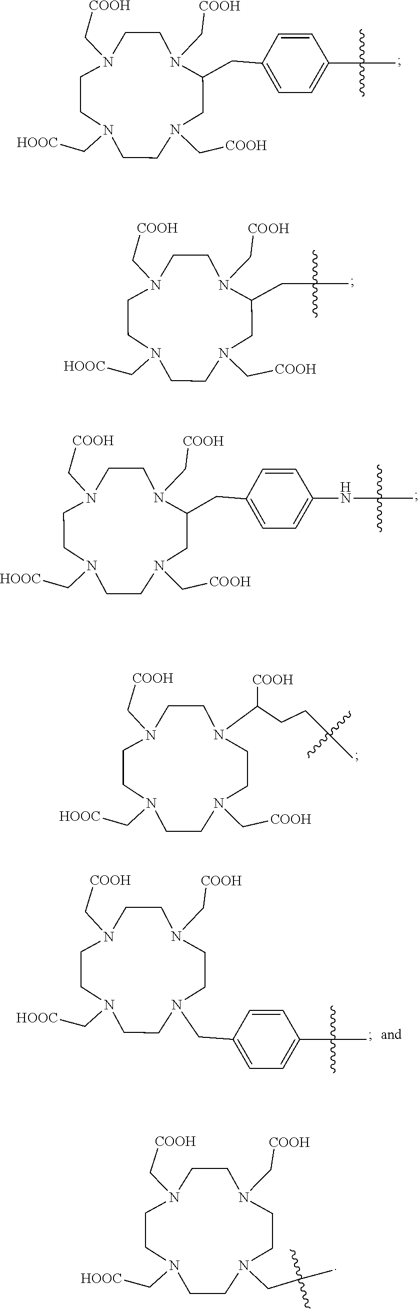 [SMILES: *c1ccc(CC2CN(CC(=O)O)CCN(CC(=O)O)CCN(CC(=O)O)CCN2CC(=O)O)cc1 ; *CC1CN(CC(=O)O)CCN(CC(=O)O)CCN(CC(=O)O)CCN1CC(=O)O ; *Nc1ccc(CC2CN(CC(=O)O)CCN(CC(=O)O)CCN(CC(=O)O)CCN2CC(=O)O)cc1 ; *CCC(C(=O)O)N1CCN(CC(=O)O)CCN(CC(=O)O)CCN(CC(=O)O)CC1 ; *c1ccc(CN2CCN(CC(=O)O)CCN(CC(=O)O)CCN(CC(=O)O)CC2)cc1 ; *CN1CCN(CC(=O)O)CCN(CC(=O)O)CCN(CC(=O)O)CC1]

According to a specific embodiment, Ch is

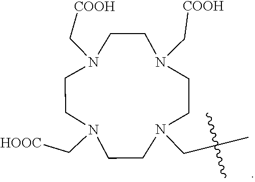 [SMILES: *CN1CCN(CC(=O)O)CCN(CC(=O)O)CCN(CC(=O)O)CC1]

According to an embodiment, W is —(C═O)—NR²—, and Ch is

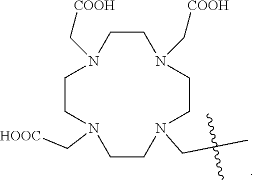 [SMILES: *CN1CCN(CC(=O)O)CCN(CC(=O)O)CCN(CC(=O)O)CC1]

According to an embodiment, m is 4, Z is COOQ, and Q is H.

In specific embodiments, according to an embodiment, R is

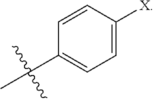

### and Ch is

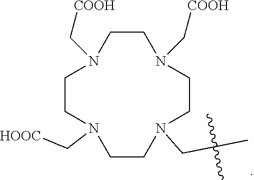 [SMILES: *CN1CCN(CC(=O)O)CCN(CC(=O)O)CCN(CC(=O)O)CC1]

According to a preferred embodiment, the PSMA binding ligand is a compound of formula (II):

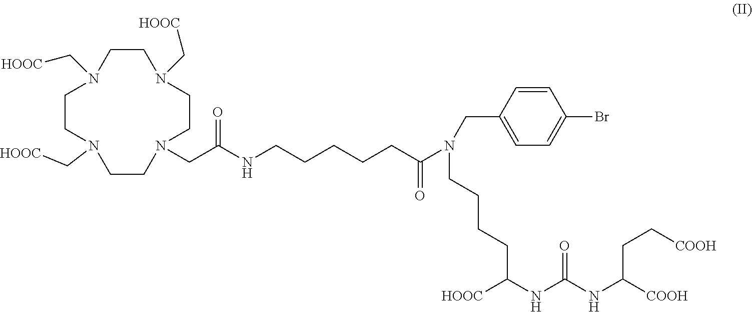 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

The compound of formula (II) can be referred to as PSMA-R2.

According to another embodiment, the PSMA binding ligand is a compound of formula (III):

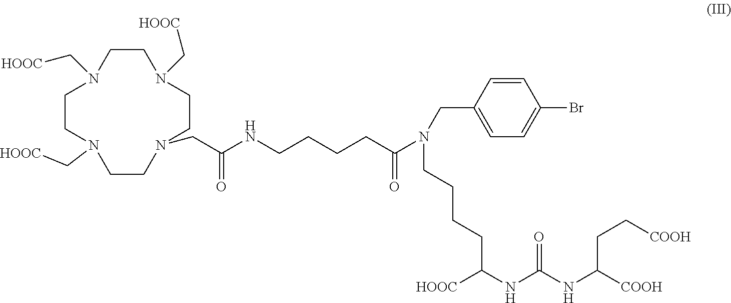 [SMILES: O=C(O)CCC(NC(=O)NC(CCCCN(Cc1ccc(Br)cc1)C(=O)CCCCNC(=O)CN1CCN(CC(=O)O)CCN(CC(=O)O)CCN(CC(=O)O)CC1)C(=O)O)C(=O)O]

The compound of formula (III) can be referred to as PSMA-Cpd2.

The First Vial Comprising said PSMA Binding Ligand

In certain embodiments, the radiolabeling method uses a single vial kit. In this embodiment, said first vial comprises said PSMA binding ligand, a buffering agent, and optionally a bulking agent, all in dried forms.

Alternatively, the radiolabeling method uses a two vial kit. In this embodiment, the first vial comprises said PSMA binding ligand, and optionally a bulking agent, and the second vial comprises the buffering agent.

For example, said PSMA binding ligand, typically PSMA binding ligand of formula (II), is comprised in said first vial at an amount between 10 and 100 μg, preferably between 15 and 60 μg, even more preferably about 30 μg.

In preferred embodiments, mannitol may be used as a bulking agent, preferably at an amount between 5 and 50 mg, preferably between 10 and 30 mg, even more preferably about 20 mg.

In a specific embodiment, the first or single does not contain an antioxidant. For example, the first or single vial does not contain gentisic acid.

A preferred example of said first vial (Vial 1 of a two vial kit) is given in the examples.

The first vial is preferably obtained by freeze-drying using methods well known in the art. Therefore, said first vial may be provided in a lyophilized or spray dried form.

As used herein, the buffering agent is a buffer suitable for obtaining a pH from 2.5 and 4.0, preferably between 2.8 and 4.0, more preferably between 3.0 and 4.0, and even more preferably between 3.2 and 3.8, at the incubating step (iii). A “buffer for a pH from 2.5 and 4.0, preferably between 2.8 and 4.0, more preferably between 3.0 and 4.0, and even more preferably between 3.2 and 3.8” may advantageously be a formic acid buffer with sodium hydroxide.

In a specific embodiment, the first or single vial does not contain an antioxidant, for example, the first or single vial does not contain gentisic acid, and the buffering agent is a buffer suitable for obtaining a pH from 2.5 and 4.0, preferably between 2.8 and 4.0, more preferably between 3.0 and 4.0, and even more preferably between 3.2 and 3.8, at the incubating step (iii).

Said buffering agent may further be comprised in the first vial, in an embodiment using a single vial kit, or in a separate second vial, in an embodiment using a two vial kit.

Step (ii) of Adding a Solution of Said Radioactive Isotope into Said Fist Vial

Radioactive isotopes for use in the radiolabeling methods include those suitable as contrast agent in PET and SPECT imaging comprising the following:

¹¹¹In, ^{133m}In, ^{99m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁵⁷Gd, ¹⁶⁹Yb, ¹⁷²Tm, ^{177m}Sn, ⁸⁹Zr, ⁴³Sc, ⁴⁴Sc, ⁵⁵Co.

According to a preferred embodiment, the radioactive isotope is ⁶⁸Ga, ⁶⁷Ga or ⁶⁴Cu. In a preferred embodiment, ⁶⁷Ga is used for SPECT imaging and ⁶⁸Ga and ⁶⁴Cu are used for PET imaging such as PET/CT or PET/MRI

The metallic ions of such radioisotopes are able to form non-covalent bond with the functional groups of the chelator, e.g. carboxylic acids of the PSMA binding ligand.

In a specific embodiment, said solution of said radioactive isotope is an eluate obtained from the steps of
i. producing a radioactive isotope from a parent non-radioactive element by means of a radioactive isotope generator,
ii. separating said radioactive isotope from said parent non-radioactive element by elution in HCl as an elution solvent, and
iii. recovering the eluate,
   thereby obtaining a solution of said radioactive isotope in HCl.

In specific embodiments, the solution containing said radioactive isotope is an aqueous solution comprising the radioisotope in the form of a metal ion, e.g. ⁶⁸Ga³⁺, ⁶⁷Ga³⁺ or ⁶⁴Cu²⁺. The solution containing said radioactive isotope can be an aqueous solution comprising ⁶⁸GaCl₃, ⁶⁷GaCl₃ or ⁶⁴CuCl₂, in HCl.

Said solution comprising the radioactive isotope ⁶⁸Ga is an eluate typically obtained from the steps of:
i. producing ⁶⁸Ga element from a parent element ⁶⁸Ge, by means of a generator, and
ii. optionally, separating the generated ⁶⁸Ga element from ⁶⁸Ge element by passing the elements ⁶⁸Ge/⁶⁸Ga through a suitable cartridge, and eluting ⁶⁸Ga in HCl,
   thereby obtaining a solution of said radioactive isotope in HCl.

Such methods of producing ⁶⁸Ga from ⁶⁸Ge/⁶⁸Ga generators are well-known in the art and for example described in Martiniova L. et al. Gallium-68 in Medical Imaging. Curr Radiopharm. 2016; 9(3):187-20; Dash A, Chakravarty Radionuclide generators: the prospect of availing PET radiotracers to meet current clinical needs and future research demands R Am J Nucl Med Mol Imaging. 2019 Feb. 15; 9(1):30-66.

Said solution comprising the radioactive isotope ⁶⁸Ga may be an eluate typically obtained from cyclotron production. Such production is for example described in Am J Nucl Med Mol Imaging 2014;4(4):303-310 or in B. J. B. Nelson et al./Nuclear Medicine and Biology 80-81 (2020) 24-31.

Typically, ⁶⁸Ga may be produced by a cyclotron, preferably using a proton beam of energy between 8 and 18 MeV, preferably between 11 and 14 MeV. The ⁶⁸Ga may be produced via the ⁶⁸Zn(p,n) ⁶⁸Ga reaction using a a solid or liquid target system. The target consists of enriched ⁶⁸Zn metal or ⁶⁸Zn liquid solution. After irradiation, the target is transferred for further chemical processing in which the ⁶⁸Ga is isolated using ion exchange chromatography. ⁶⁸Ga is eluted in HCl solution.

Alternatively, said radioactive isotope is ⁶⁷Ga. Various methods for the production of ⁶⁷Ga, using either a zinc (enriched or natural) or copper or germanium target with protons, deuterons, alpha particles or helium(III) as the bombarding particle, have been reported as summarised by Helus, F., Maier-Borst, W., 1973. A comparative investigation of methods used to produce ⁶⁷Ga with a cyclotron. In: Radiopharmaceuticals and Labelled Compounds, Vol. 1, IAEA, Vienna, pp. 317-324, M.L Thakur Gallium-67 and indium-111 radiopharmaceuticals Int. J. Appl. Rad. Isot., 28 (1977), pp. 183-201, and Bjørnstad, T., Holtebekk, T., 1993. Production of ⁶⁷Ga at Oslo cyclotron. University of Oslo Report OUP8-3-1, pp. 3-5. Bombardment of natGe targets with moderate energy protons (up to 64 MeV) is also a suitable method to produce ⁶⁷Ga as described in T Horiguchi, H Kumahora, H Inoue, Y Yoshizawa Excitation functions of Ge(p,xnyp) reactions and production of ⁶⁸Ge, Int. J. Appl. Radiat. Isot., 34 (1983), pp. 1531-1535.

Preferably, ⁶⁷Ga may be produced by a cyclotron. Such methods of producing ⁶⁷Ga from ⁶⁸Zn (p, 2n) ⁶⁷Ga are well-known in the art and for example described inAlirezapour B et al. Iranian Journal of Pharmaceutical Research (2013), 12 (2): 355-366. More preferably, this method uses a proton beam of energy between 10 and 40 MeV. The ⁶⁷Ga may be produced via either the ⁶⁷Zn (p, n) ⁶⁷Ga or either the ⁶⁸Zn (p, 2n) ⁶⁷Ga reaction using a solid or liquid target system. The target consisted of enriched ⁶⁷Zn or ⁶⁸Zn metal or liquid solution. After irradiation, the target is transferred for further chemical processing in which the ⁶⁷Ga is isolated using ion exchange chromatography. Final evaporation from aq. HCl yield ⁶⁷GaCl₃, which may then be added to said single vial for the labelling method.

Alternatively, said radioactive isotope is ⁶⁴Cu as obtained from cyclotron production. Such production method is for example described in WO2013/029616.

Typically, ⁶⁴Cu may be produced by a cyclotron, preferably using a proton beam of energy between 11 and 18 MeV. The ⁶⁴Cu may be produced via the ⁶⁴Ni (p,n) ⁶⁴Cu reaction using a solid or liquid target system. The target consisted of ⁶⁴Ni metal or ⁶⁴Ni liquid solution. After irradiation, the target is transferred for further chemical processing in which the ⁶⁴Cu is isolated using ion exchange chromatography. Final evaporation from aq. HCl yield ⁶⁴CuCl₂, which may then be added to said first vial for the labelling method.

Step (iii) of mixing the solution obtained in step (ii) with at least a buffering agent, and incubating it for a sufficient period of time for obtaining said PSMA binding ligand labeled with said radioactive isotope. Step (iii) is preferably performed at sufficiently elevated temperature, for example at least 50° C., and preferably between 50° C. and 100° C.

The radiolabelling starts after the mixing of first vial comprising the PSMA binding ligand (e.g; the PSMA binding ligand of formula (II)) with the solution comprising the radioactive isotope (typically, ⁶⁸Ga, ⁶⁷Ga or ⁶⁴Cu as disclosed above) in a suitable buffering agent as disclosed above.

In specific embodiments, the incubating step is performed at a temperature between 50° C. to 100° C. In specific embodiments, the incubating step is performed for a period of time comprised between 2 and 25 minutes.

In specific embodiments, the incubating step is performed at a temperature between 80° C. and 100° C., preferably between 90° C. and 100° C., typically at about 95° C.

In other specific embodiments, the incubating step is performed at a temperature between 50° C. and 90° C., preferably between 60° C. and 80° C., typically at about 70° C.

In specific embodiments, the incubating step is performed for a period of time comprised between 2 and 20 minutes, preferably between 5 and 10 minutes, preferably between 6 and 8 minutes, even more preferably about 7 minutes.

In other specific embodiments, the incubating step is performed for a period of time comprised between 5 and 25 minutes, preferably between 10 and 20 minutes, preferably between 12 and 18 minutes, even more preferably about 15 minutes.

At the end of labeling process, a sequestering agent having a particular affinity for the radioactive isotope (such as ⁶⁸Ga, ⁶⁷Ga or ⁶⁴Cu) may be added to chelate the non-reacted part of the isotope. This complex formed by the sequestering agent and the non-reacted radioactive isotope may then be discarded to increase the radiochemical purity after radiolabelling.

### Preferred Embodiments of the Methods for Radiolabelling PSMA Binding Ligand of Formula (II) with ⁶⁸Ga

The present disclosure more particularly relates to a method for labeling a PSMA binding ligand of formula (II)

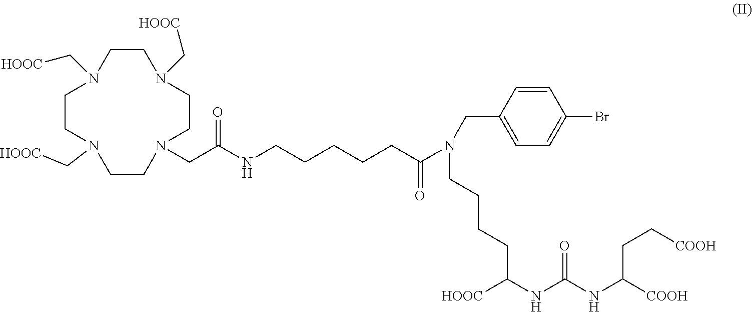 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

with ⁶⁸Ga, comprising the steps of:
   i. providing a first vial containing about 30 μg of PSMA binding ligand of formula (II), in dried form,
   ii. adding a solution of ⁶⁸Ga in HCl to said first vial,
   iii. mixing the solution obtained in ii. with a reaction solution comprising a buffering agent for adjusting the pH in a range of 3.2 and 3.8, and incubating it for a sufficient period of time and at sufficiently elevated temperatures for obtaining said PSMA binding ligand labeled with ⁶⁸Ga, and
   iv. optionally adjusting the pH of the solution.

In specific embodiments of said methods, said solution of said ⁶⁸Ga in HCl is an eluate obtained from the steps of
i. producing ⁶⁸Ga element from a parent element ⁶⁸Ge, by means of a generator, and
ii. optionally, separating the generated ⁶⁸Ga element from ⁶⁸Ge element by passing the elements ⁶⁸Ga/⁶⁸Ge through a suitable cartridge, and eluting ⁶⁸Ga in HCl,
   thereby obtaining a solution of said radioactive isotope in HC.

Typically, said buffering agents consist of 60 mg of formic acid and 56.5 mg of sodium hydroxide.

In a specific embodiment, the powder for solution for injection does not contain an antioxidant. For example, the powder for solution for injection does not contain gentisic acid.

Advantageously, in specific embodiments, a simple labelling of the PSMA binding ligand may be obtained with an eluate of ⁶⁸Ga in HCl coming from commercially available ⁶⁸Ge/⁶⁸Ga generators without any processing of the eluate or any additional purification step.

Powder for a solution for injection The disclosure further relates to a powder for solution for injection, comprising the following components in dried forms:
i. a PSMA binding ligand of formula (I):

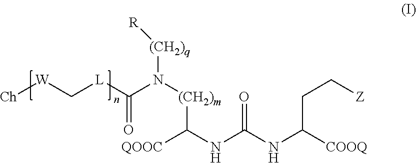

wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X; X is -V-Y;
V is a bond or a C₁-C₆ alkylene, preferably V is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′″, —OC(O)NR′R″, —NR″C(O)R′, —NR′—C(O)NR″R′″, —NR″C(O)OR′, —NR′—C(NR″R′″)═NR″″, —S(O)R′, —S(O)₂R′, —S(O)₂NR′R″, —NRSO₂R′, —CN and —NO₂ in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR₂—(C═O), —NR²—(C═S), —(C═O)—NR²—, and —(C═S)—NR₂—, preferably, W is —(C═O)—NR₂—;
each occurrence of L and W can be the same or different;
R² is H or C₁-C₄ alkyl, preferably R² is H;
n is an integer selected from the group consisting of 1, 2 and 3;
Ch is a chelating agent, typically DOTA; and
   ii. a bulking agent, for example, mannitol.

A preferred embodiment comprises the following components:
i. a PSMA binding ligand of formula (II) in an amount between 10 and 100 μg, preferably between 15 and 60 μg, even more preferably about 30 μg;

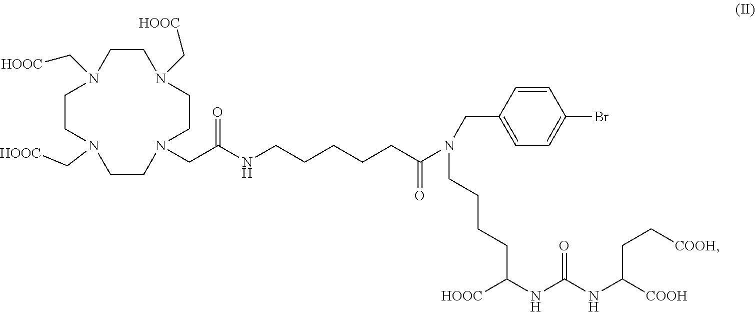 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. mannitol in an amount between 5 and 50 mg, preferably between 10 and 30 mg, even more preferably about 20 mg.

In a specific embodiment, the powder for solution for injection does not contain an antioxidant. For example, the powder for solution for injection does not contain gentisic acid.

### Radiolabelling Kits of the Disclosure

The present disclosure also relates to a kit for carrying out the above labeling methods, said kit comprising
i. a first vial with the following components in dried forms
   i. a PSMA binding ligand of formula (II):

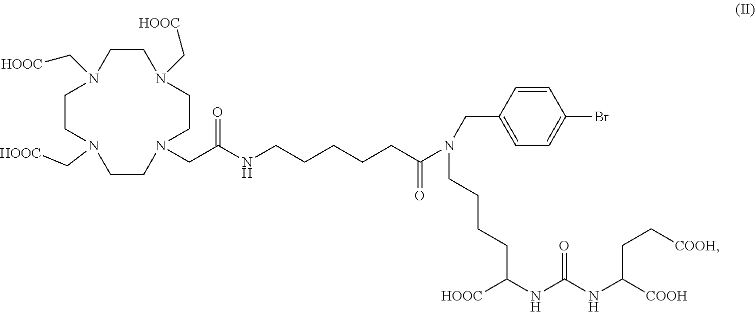 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. optionally a bulking agent, for example, mannitol, and,
ii. a second vial comprising at least a buffering agent, preferably in dried form; and,
iii. optionally, an accessory cartridge for eluting a radioactive isotope generated by a radioactive isotope generator or a cyclotron.

Preferably, said first or single vial comprises the following components:
i. a PSMA binding ligand of formula (II) in an amount between 10 and 100 μg, preferably between 15 and 60 μg, even more preferably about 30 μg;

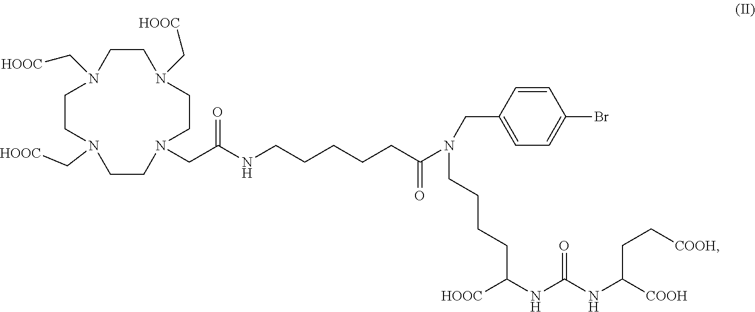 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. mannitol in an amount between 5 and 50 mg, preferably between 10 and 30 mg, even more preferably about 20 mg.

Said second vial or single vial may comprise buffering agents for maintaining a pH between 2.5 and 4.0, preferably between 2.8 and 4.0, more preferably between 3.0 and 4.0, and even more preferably between 3.2 and 3.8. For example, said second vial comprises formic acid and sodium hydroxide as buffering agents. The buffering agents can be in dried form or in solution. According to an embodiment, said buffering agents consist of an aqueous solution of formic acid and sodium hydroxide, wherein formic acid is present at a concentration of about 60 mg/mL and sodium hydroxide is present at a concentration of about 56.5 mg/mL.

Preferably, all components of said first, second or single vial are in dried forms.

The radioactive isotope for labeling the PSMA binding ligand may be provided with the kit as ready-for-use product, i.e. for mixing and incubating with the first vial and buffering agent as provided by the kit, or alternatively may be eluted from a radioactive isotope generator or a cyclotron prior to, and shortly before mixing and incubating with said first vial and buffering agent, particularly in cases said radioactive isotope has a relatively short half-life such as ⁶⁸Ga, ⁶⁷Ga and ⁶⁴Cu. The radioactive isotope for labeling, such as ⁶⁸Ga, ⁶⁷Ga or ⁶⁴Cu, may also be produced by a cyclotron.

Preferably, the components are inserted into sealed containers which may be packaged together, with instructions for performing the method according to the present disclosure.

The kit can also be used as a part of an automatic system or a remotely controlled mechanism system that automatically performs the elution of the gallium-68 generator and/or the subsequent mixing and heating. In this embodiment, the vial containing the PSMA binding ligand (first vial) is directly connected to the elution system and/or the heating system

The kit may be applied in particular for use in the methods as disclosed in the next section.

In a specific embodiment, the kit does not contain an antioxidant. For example, the kit does not contain gentisic acid.

In a specific embodiment, the kit does not contain an antioxidant, for example, the kit does not contain gentisic acid, and said second or single vial comprises buffering agents for maintaining a pH between 2.5 and 4.0, preferably between 2.8 and 4.0, more preferably between 3.0 and 4.0, and even more preferably between 3.2 and 3.8.

In specific embodiments, the PSMA binding ligand is the PSMA binding ligand of formula (II) as defined above.

### Use of the Kit According to the Present Disclosure

The above-defined kits may be applied in particular for use of the labeling methods as disclosed in the previous sections.

Advantageously, a solution comprising a PSMA binding ligand (e.g. PSMA binding ligand of formula (II)) labeled with a radioactive isotope (for example ⁶⁸Ga, ⁶⁷Ga or ⁶⁴Cu) is obtainable or obtained by the labeling methods as disclosed in the previous sections.

Such solution may be ready for use as an injectable solution, for example, for in vivo detection of tumors by imaging in a subject in need thereof.

In certain aspects the subject is a mammal, for example but not limited to a rodent, canine, feline, or primate. In preferred aspects, the subject is a human.

The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and SHP Handbook on Injectable Drugs, Trissel, 15th ed., pages 622-630 (2009)).

Typically, said solution for use as an injectable solution provides a single dose between 100-350 MBq, preferably between 150-250 MBq of [⁶⁸Ga]-PSMA binding ligand of formula (II) for administration to a subject in need thereof.

In specific embodiments, said subject in need thereof is a subject that has a cancer having PSMA expressing tumor or cells. The PSMA-expressing tumor or cell can be selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof. In some other embodiments, the PSMA-expressing tumors or cells is a prostate tumor or cell

Typically, PET/MRI, SPECT or PET/CT imaging may be acquired 20 to 120 minutes preferably between 50 to 100 minutes after the intravenous administration of the radiolabelled PSMA binding ligand to the subject, and more preferably with 2 and 3 hours after the administration of the radiolabelled PSMA binding ligand to the subject.

### Synthesis of the Compounds of Formula (I), (II) and (III)

The compounds of formula (I), (II) and (III) can be synthesized using the methods disclosed in WO2017/165473.

In particular, the compound of formula (II) can be synthesized as disclosed in scheme 1. The p-bromobenzyl group modified of Glu-Lys urea 2 can be prepared by reductive alkylation of Glu-Lys urea 1 with p-bromobenzaldehyde in presence of sodium cyanoborohydride in methanol. This procedure has been described in the literature (Tykvart et al. (2015) Journal of medicinal chemistry 58, 4357-63). Then, an aliphatic linker, Boc-6-aminohexanoic acid can be coupled on the same c-Lys amine of 2, for example using a base (like N,N-diisopropylethylamine) and a coupling agent (like N,N,N′,N′-Tetramethyl-O-(N-succinimidyl)uronium tetrafluoroborate or 1-[Bis(dimethylamino)methylene]-1H-1,2,3-triazolo[4,5-b]pyridinium-3-oxid hexafluoro-phosphate), to yield compound 3. Compound 3 can then be deprotected to yield compound 4, for example using an acid like trifluoroacetic acid. Finally, conjugation with commercially available DOTA-NHS ester can be performed to yield compound (II).

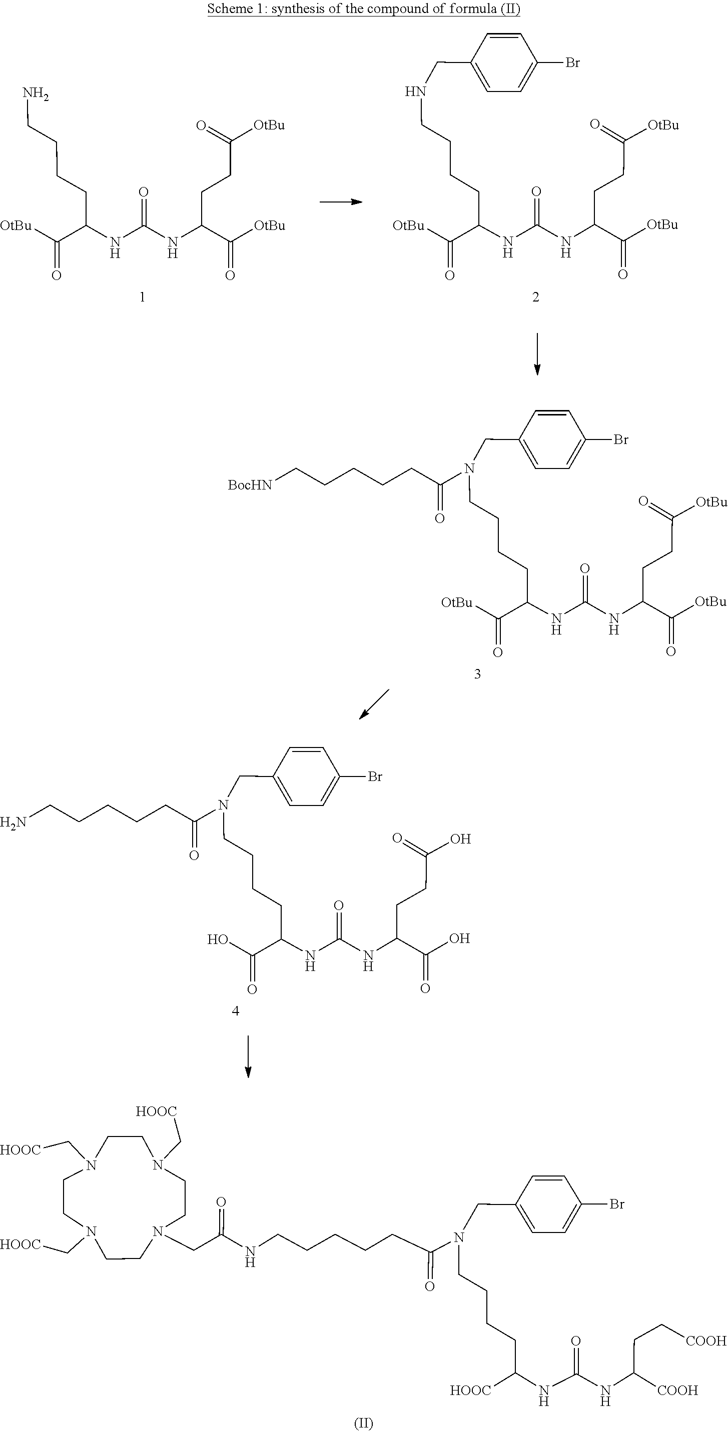 [SMILES: CC(C)(C)OC(=O)CCC(NC(=O)NC(CCCCN)C(=O)OC(C)(C)C)C(=O)OC(C)(C)C ; CC(C)(C)OC(=O)CCC(NC(=O)NC(CCCCNCc1ccc(Br)cc1)C(=O)OC(C)(C)C)C(=O)OC(C)(C)C ; CC(C)(C)OC(=O)CCC(NC(=O)NC(CCCCN(Cc1ccc(Br)cc1)C(=O)CCCCCNC(=O)OC(C)(C)C)C(=O)OC(C)(C)C)C(=O)OC(C)(C)C ; NCCCCCC(=O)N(CCCCC(NC(=O)NC(CCC(=O)O)C(=O)O)C(=O)O)Cc1ccc(Br)cc1 ; O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O ; "(II)"]

### Embodiments

The following specific embodiments are disclosed:
1. A method for labeling a PSMA binding ligand with a radioactive isotope, preferably ⁶⁸Ga, ⁶⁷Ga or ⁶⁴Cu, said method comprising the steps of:
   i. providing a first vial comprising said PSMA binding ligand, and optionally a bulking agent, in dried form,
   ii. adding a solution of said radioactive isotope into said first vial, thereby obtaining a solution of said PSMA binding ligand with said radioactive isotope,
   iii. mixing the solution obtained in ii. with at least a buffering agent and incubating it for a sufficient period of time for obtaining said PSMA binding ligand labeled with said radioactive isotope, and,
   iv. optionally, adjusting the pH of the solution.
2. The method of Embodiment 1, wherein the first vial at step i. is a vial comprising said PSMA binding ligand, a buffering agent, and optionally a bulking agent, preferably all in dried forms.
3. The method of Embodiment 1, wherein step iii comprises mixing the solution obtained in ii. with at least a reaction solution comprising a buffering agent and incubating it for a sufficient period of time and at sufficiently elevated temperatures for obtaining said PSMA binding ligand labeled with said radioactive isotope.
4. The method of any one of Embodiments 1-3, wherein said solution with said radioactive isotope further comprises HC1.
5. The method of any one of Embodiments 1-4, wherein said radioactive isotope is ⁶⁸Ga and the radiochemical purity as measured in HPLC is at least 92%, and optionally, the percentage of free ⁶⁸Ga³⁺ (in HPLC) is 2% or less, and/or the percentage of non-complexed ⁶⁸Ga³⁺ species (in ITLC) is 3% or less.
6. The method of any one of Embodiments 1-4, wherein said radioactive isotope is ⁶⁴Cu and the radiochemical purity as measured in HPLC is at least 92%, and optionally, the percentage of free ⁶⁴Cu²⁺ (in HPLC) is 2% or less, and/or the percentage of non-complexed ⁶⁴Cu²⁺ species (in ITLC) is 3% or less.
7. The method of any one of Embodiments 1-4, wherein said radioactive isotope is 67Ga and the radiochemical purity as measured in HPLC is at least 92%, and optionally, the percentage of free ⁶⁷Ga3+ (in HPLC) is 2% or less, and/or the percentage of non-complexed ⁶⁷Ga³⁺ species (in ITLC) is 3% or less.
8. The method of any one of Embodiments 1-7, wherein said PSMA binding ligand is a compound of formula (I):

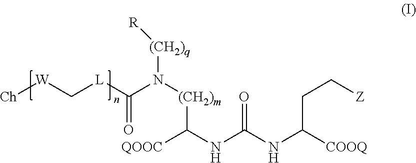

wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is -V-Y;
V is a bond or a C₁-C₆ alkylene, preferably V is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′″)═NR″″)═NR″″, —S(O)R′, —S(O)₂R′, —S(L)₂NR′R″, —NRSO₂R′, —CN and —NO₂ in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR₂—(C═O), —NR²—(C═S), —(C═O)—NR²—, and —(C═S)—NR₂—, preferably, W is —(C═O)—NR₂—;
each occurrence of L and W can be the same or different;
R2 is H or C₁-C₄ alkyl, preferably R² is H;
n is an integer selected from the group consisting of 1, 2 and 3;
Ch is a chelating agent, typically DOTA.
9. The method of Embodiment 8, wherein said PSMA binding ligand is a compound of formula (II):

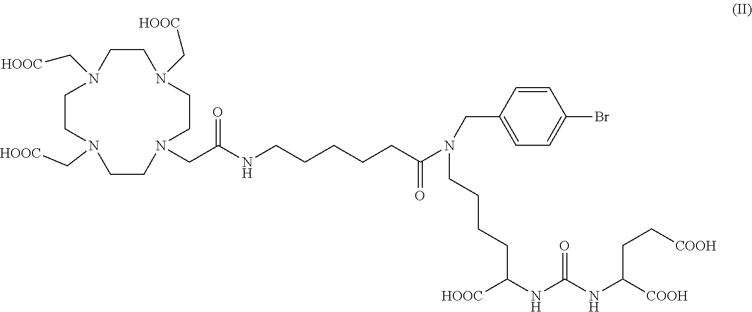 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

10. The method of any one of Embodiments 1-9, wherein said PSMA binding ligand is comprised in said first vial in an amount between 10 and 100 μg, preferably between 15 and 60 μg, even more preferably about 30 μg.
11. The method of any one of Embodiments 1-10, wherein said first vial further comprises mannitol as a bulking agent, preferably between 5 and 50 mg, preferably between 10 and 30 mg, even more preferably about 20 mg.
12. The method of any one of Embodiments 1-11, wherein said buffering agent is present in an amount suitable for obtaining a pH between 2.5 and 4.0, preferably between 2.8 and 4.0, more preferably between 3.0 and 4.0, and even more preferably between 3.2 and 3.8, at the incubating step (iii).
13. The method of any one of Embodiments 1-12, wherein said buffering agent comprises formic acid and sodium hydroxide as buffering agents.
14. The method of any one of Embodiments 1-13, wherein the incubating step is performed at a temperature between 50° C. and 100° C.
15. The method of any one of Embodiments 1-14, wherein the incubating step is performed for a period of time comprised between 2 and 25 minutes.
16. The method of any one of Embodiments 1-15, wherein the incubating step is performed at a temperature between 80° C. and 100° C., preferably between 90° C. and 100° C., even more preferably about 95° C.
17. The method of any one of Embodiments 1-16, wherein the incubating step is performed for a period of time comprised between 2 and 20 minutes, preferably between 5 and 10 minutes, preferably between 6 and 8 minutes, even more preferably about 7 minutes.
18. The method of any one of Embodiments 1-15, wherein the incubating step is performed at a temperature 50° C. and 90° C., preferably between 60° C. and 80° C., typically at about 70° C.
19. The method of any one of Embodiments 1-15 or 18, wherein the incubating step is performed for a period of time comprised between 5 and 25 minutes, preferably between 10 and 20 minutes, preferably between 12 and 18 minutes, even more preferably about 15 minutes.
20. The method of any one of Embodiments 1-19, wherein said solution of said radioactive isotope is an eluate obtained from the steps of
   i. producing a radioactive isotope from a parent non-radioactive element by means of a radioactive isotope generator,
   ii. separating said radioactive isotope from said parent non-radioactive element by elution,
   iii. recovering the eluate, thereby obtaining a solution of said radioactive isotope.
21. The method of any one of Embodiments 1-19, wherein said solution of said radioactive isotope is an eluate obtained from the steps of
   i. producing a radioactive isotope from a non-radioactive or radioactive element by means of a cyclotron,
   ii. separating said radioactive isotope from said non-radioactive or radioactive element by elution,
   iii. recovering the eluate, thereby obtaining a solution of said radioactive isotope.
22. The method of any one of Embodiments 1-21, wherein the first or single vial does not contain an antioxidant, for example, the first or single vial does not contain gentisic acid, and the buffering agent is a buffer suitable for obtaining a pH from 2.5 and 4.0, preferably between 2.8 and 4.0, more preferably between 3.0 and 4.0, and even more preferably between 3.2 and 3.8, at the incubating step (iii).
23. The method of any one of Embodiments 1-22, wherein said first or single vial does not contain gentisic acid.
24. A method for labeling a PSMA binding ligand of formula (II)

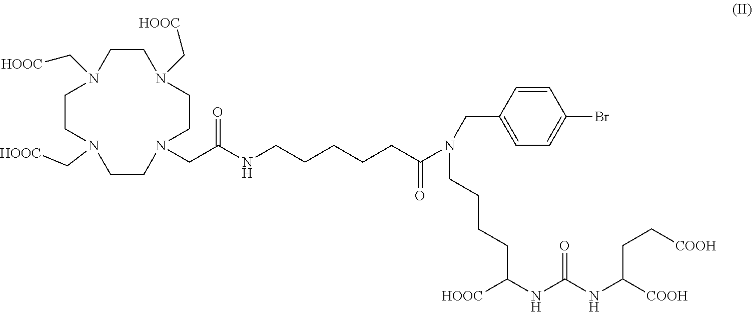 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

with ⁶⁸Ga, comprising the steps of:
i. providing a first vial containing about 30 μg of PSMA binding ligand of formula (II), in dried form,
ii. adding a solution of ⁶⁸Ga in HCl to said first vial,
iii. mixing the solution obtained in ii. with a reaction solution comprising a buffering agent for adjusting the pH in a range of 3.2 and 3.8, and incubating it for a sufficient period of time and at sufficiently elevated temperatures for obtaining said PSMA binding ligand labeled with ⁶⁸Ga,
iv. optionally adjusting the pH of the solution.
25. The method of Embodiment 24, wherein said solution of said ⁶⁸Ga in HCl is an eluate obtained from the steps of
   i. producing ⁶⁸Ga element from a parent element ⁶⁸Ge, by means of a generator,
   ii. separating the generated ⁶⁸Ga element from ⁶⁸Ge element by passing the elements ⁶⁸Ga/⁶⁸Ge through a suitable cartridge, and eluting ⁶⁸Ga in HCl, thereby obtaining a solution of said radioactive isotope in HCl.
26. The method of Embodiment 24, wherein said solution of said ⁶⁸Ga in HCl is an eluate obtained from the steps of
   i. producing ⁶⁸Ga element from an element, e.g. ⁶⁸Zn, by means of a cyclotron,
   ii. separating the generated ⁶⁸Ga element from the starting element by passing the elements ⁶⁸Ga/starting element through a suitable cartridge, and eluting ⁶⁸Ga in HCl,
   thereby obtaining a solution of said radioactive isotope in HCl.
27. A method for labeling a PSMA binding ligand of formula (II)

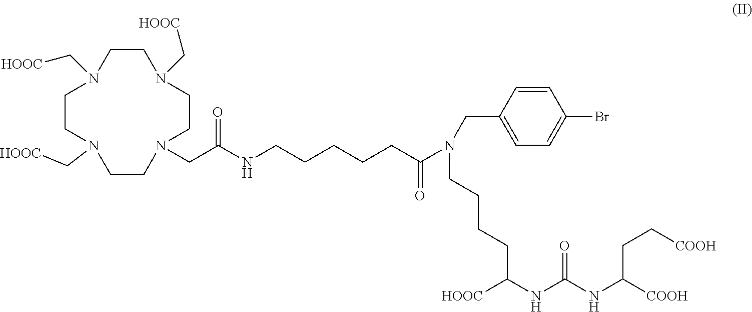 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

with ⁶⁷Ga, comprising the steps of:
i. providing a first vial containing about 30 μg of PSMA binding ligand of formula (II), in dried form,
ii. adding a solution of ⁶⁷Ga in HCl to said first vial,
iii. mixing the solution obtained in ii. with a reaction solution comprising a buffering agent for adjusting the pH in a range of 3.2 and 3.8, and incubating it for a sufficient period of time and at sufficiently elevated temperatures for obtaining said PSMA binding ligand labeled with ⁶⁷Ga,
optionally adjusting the pH of the solution.
28. A method for labeling a PSMA binding ligand of formula (II)

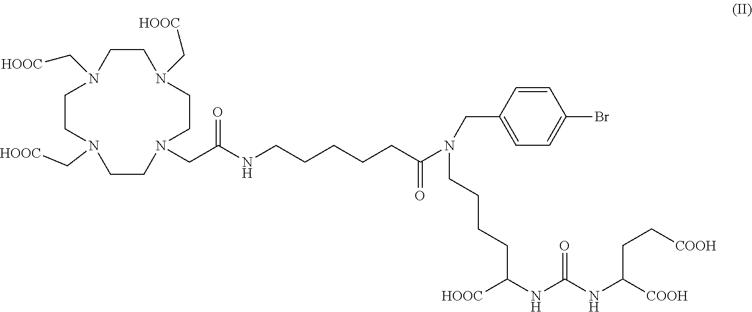 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

with ⁶⁴Cu, comprising the steps of:
i. providing a first vial containing about 30 μg of PSMA binding ligand of formula (II), in dried form,
ii. adding a solution of ⁶⁴Cu in HCl to said first vial,
iii. mixing the solution obtained in ii. with a reaction solution comprising a buffering agent for adjusting the pH in a range of 3.2 and 3.8, and incubating it for a sufficient period of time and at sufficiently elevated temperatures for obtaining said PSMA binding ligand labeled with ⁶⁴Cu,
iv. optionally adjusting the pH of the solution.
29. The method of Embodiment 24-28, wherein the first or single vial does not contain an antioxidant, for example, the first or single vial does not contain gentisic acid, and the buffering agent is a buffer suitable for obtaining a pH from 2.5 and 4.0, preferably between 2.8 and 4.0, more preferably between 3.0 and 4.0, and even more preferably between 3.2 and 3.8, at the incubating step (iii).
30. The method of any one of Embodiments 24-29, wherein said buffering agents consist of 60 mg of formic acid and 56.5 mg of sodium hydroxide.

31. The method of any one of Embodiments 24-29, wherein said buffering agents consist of an aqueous solution of formic acid and sodium hydroxide, wherein formic acid is present at a concentration of about 60 mg/mL and sodium hydroxide is present at a concentration of about 56.5 mg/mL.
32. The method of any one of Embodiments 24-31, wherein the incubating step is performed at a temperature between 50° C. and 100° C.
33. The method of any one of Embodiments 24-32, wherein the incubating step is performed for a period of time comprised between 2 and 25 minutes.
34. The method of any one of Embodiments 24-27 and 29-33, wherein the incubating step is performed at a temperature between 80° C. and 100° C., preferably between 90° C. and 100° C., typically at about 95° C.
35. The method of any one of Embodiments 24-27 or 29-34, wherein the incubating step is performed for a period of time comprised between 2 and 20 minutes, preferably between 5 and 10 minutes, preferably between 6 and 8 minutes, even more preferably about 7 minutes.

36. The method of any one of Embodiments 28-33, wherein the incubating step is performed at a temperature between 50° C. and 90° C., preferably between 60° C. and 80° C., typically at about 70° C.

37. The method of any one of Embodiments 28-33 or 36, wherein the incubating step is performed for a period of time comprised between 5 and 25 minutes, preferably between 10 and 20 minutes, preferably between 12 and 18 minutes, even more preferably about 15 minutes.
38. The method of any one of Embodiments 24-37, wherein said first or single vial does not contain an antioxidant, e.g. gentisic acid.
39. A solution comprising a PSMA binding ligand labeled with a radioactive isotope, obtainable or obtained by the method of any one of Embodiments 1-23, for use as an injectable solution for in vivo detection of tumors, typically PSMA-expressing tumors, by imaging in a subject in need thereof.
40. A solution according to embodiment 39 wherein the radioactive isotope is selected from the group consisting of ¹¹¹In, ^{133m}In, ⁹⁹mTc, ⁹⁴mTc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁵⁷Gd, ¹⁶⁹Yb, ¹⁷²Tm, ^{177m}Sn, ⁸⁹Zr, ⁴³Sc, ⁴⁴Sc, ⁵⁵Co.
41. A solution comprising PSMA binding ligand of formula (II) labeled with ⁶⁸Ga, ⁶⁷Ga or ⁶⁴Cu, obtainable or obtained by the method of any one of Embodiments 24-38, for use as an injectable solution for in vivo detection of tumors, typically PSMA-expressing tumors, by imaging in a subject in need thereof.
42. A powder for solution for injection, comprising the following components in dried forms:
   i. a PSMA binding ligand of formula (I):

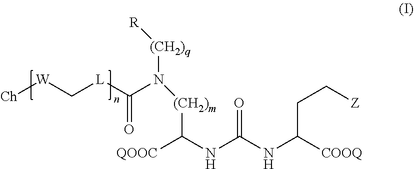

wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is -V-Y;
V is a bond or a C₁-C₆ alkylene, preferably V is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′C(NR″R′″)═NR″″, —S(O)R′, —S(O)₂R′, —S(O)₂NR′R″, —NRSO₂R′, —CN and —NO₂in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR₂—(C═O), —NR₂—(C═S), —(C═O)—NR₂—, and —(C═S)—NR₂—, preferably, W is —(C═O)—NR₂—;
each occurrence of L and W can be the same or different;
R² is H or C₁-C₄ alkyl, preferably R² is H;
n is an integer selected from the group consisting of 1, 2 and 3;
Ch is a chelating agent, typically DOTA; and
ii. a bulking agent, for example, mannitol.
43. The powder for solution for injection of Embodiment 42, wherein said PSMA binding ligand is of formula (II):

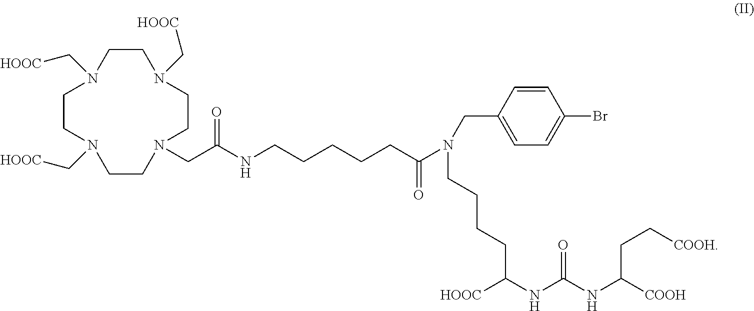 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

44. The powder for solution for injection of Embodiment 42 or 43, wherein the PSMA binding ligand is comprised in an amount between 10 and 100 μg, preferably between 15 and 60 μg, even more preferably about 30 μg.
45. The powder for solution for injection of any of Embodiments 42-44, wherein said bulking agent is mannitol in an amount between 5 and 50 mg, preferably between 10 and 30 mg, even more preferably about 20 mg.
46. The powder for solution for injection of any of Embodiments 42-45, comprising the following components:
   i. a PSMA binding ligand of formula (II) in an amount between 10 and 100 μg, preferably between 15 and 60 μg, even more preferably about 30 μg;

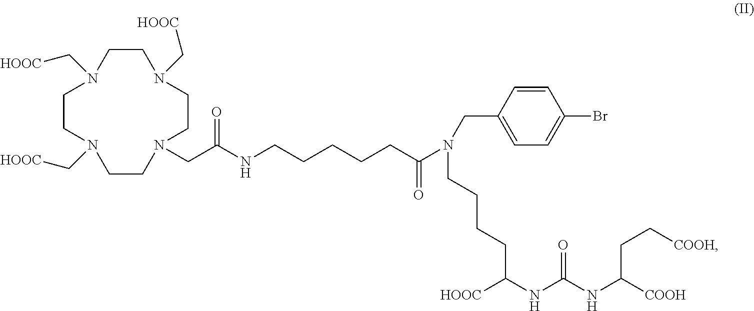 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. mannitol in an amount between 5 and 50 mg, preferably between 10 and 30 mg, even more preferably about 20 mg.
47. The powder for solution for injection of any of Embodiments 42-46, wherein said powder does not contain an antioxidant, for example the powder does not contain gentisic acid.
48. A kit for carrying out the method of any of Embodiment 24-28, comprising
   i. a first vial with the following components in dried forms
      i. a PSMA binding ligand of formula (II):

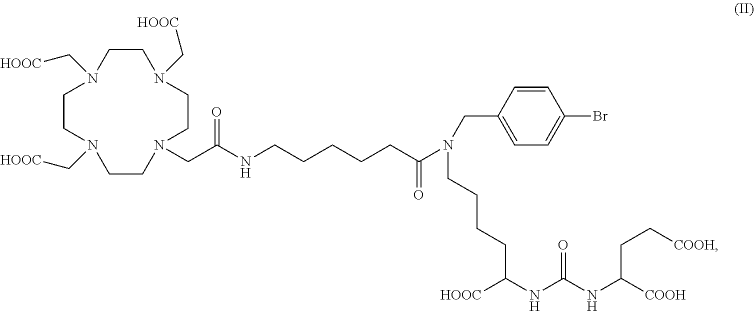 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. optionally a bulking agent, for example, mannitol, and,
ii. a second vial comprising at least a buffering agent, preferably in dried form; and,
iii. optionally, an accessory cartridge for eluting a radioactive isotope generated by a radioactive isotope generator or a cyclotron.
49. A kit for carrying out the method of any of Embodiment 24-28, comprising
   i. a single vial with the following components, preferably in dried forms:
      i. a PSMA binding ligand of formula (II):

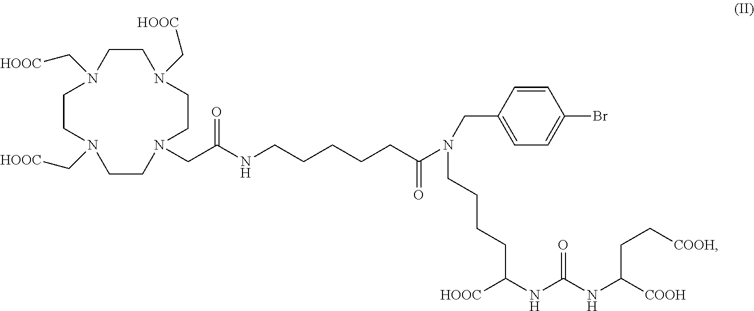 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. optionally a bulking agent, for example, mannitol,
iii. at least a buffering agent, and,
ii. optionally, an accessory cartridge for eluting a radioactive isotope generated by a radioactive isotope generator or a cyclotron.
50. The kit of Embodiment 48 or 49, wherein the PSMA binding ligand is comprised in an amount between 10 and 100 μg, preferably between 15 and 60 μg, even more preferably about 30 μg.
51. The kit of any one of Embodiments 48-50, wherein said bulking agent is mannitol in an amount between 5 and 50 mg, preferably between 10 and 30 mg, even more preferably about 20 mg.
52. The kit of any one of Embodiments 48-51, wherein said first or single vial comprises the following components:
   i. a PSMA binding ligand of formula (II) in an amount between 10 and 100 μg, preferably between 15 and 60 μg, even more preferably about 30 μg;

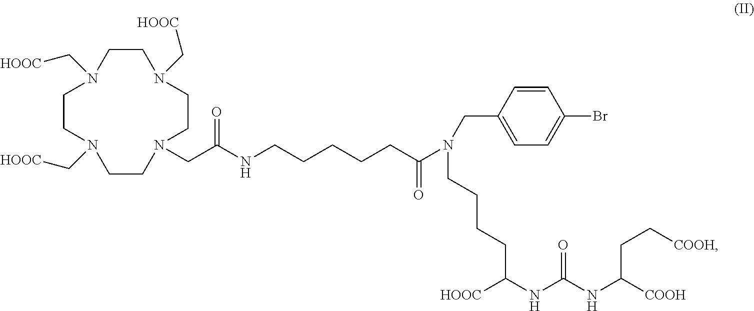 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

and
ii. mannitol in an amount between 5 and 50 mg, preferably between 10 and 30 mg, even more preferably about 20 mg.
53. The kit of any one of Embodiments 48-52, wherein said second or single vial comprises buffering agents for maintaining a pH between 2.5 and 4.0, preferably between 2.8 and 4.0, more preferably between 3.0 and 4.0, and even more preferably between 3.2 and 3.8.
54. The kit of any one of Embodiments 48-53, wherein the kit does not contain an antioxidant, for example, the kit does not contain gentisic acid, and said second vial or single vial comprises buffering agents for maintaining a pH between 2.5 and 4.0, preferably between 2.8 and 4.0, more preferably between 3.0 and 4.0, and even more preferably between 3.2 and 3.8.
55. The kit of any one of Embodiments 48-54, wherein said first or single vial does not contain gentisic acid.
56. The kit any one of Embodiments 48-55, wherein said second or single vial comprises formic acid and sodium hydroxide as buffering agents.
57. The kit of any one of Embodiments 48-56, wherein all components of said first, second or single vial are in dried forms.

### EXAMPLES

Hereinafter, the present disclosure is described in more details and specifically with reference to the examples, which however are not intended to limit the present invention.

Radiochemical purity: Non-complexed ⁶⁸Ga species by ITLC

Preparation of the mobile phase solutions:

Ammonium acetate 5M: Accurately weigh 3.85 g of ammonium acetate in a graduate flask of 10 mL and dissolve it with 10 mL of MilliQ water.

Ammonium acetate/MeOH: Using a graduated cylinder, add 1 mL of the ammonium acetate solution 5 M, 4 mL of MilliQ water and 5 mL of methanol. Transfer the eluent in the TLC chamber.

ITLC-SG preparation: Cut one ITLC-SG of 115 mm per each vial, draw a line at 10 mm from the bottom (where put a 5 uL drop of sample) and draw a line at 105 mm from the bottom (where the chromatographic development must give up).

⁶⁸Ga-PSMA-R2: reference factor 0.7-1.0

⁶⁸Ga non-complexed species: reference factor=0.0∓0.1

(⁶⁸Ga non-complexed species refers to ⁶⁸Ga colloidal species and ⁶⁸Ga free.)

Radiochemical purity and identification of ⁶⁸GaPSMA-R2 by HPLC

### Chromatographic conditions

| Flow rate | 1.0 mL/min | | | |
|---|---|---|---|---|
| Column type | Aeris PEPTIDE 3.6u XB-C18, 150 × 4.6 mm | | | |
| | Security Guard Ultra Cartridge UHPLC C18 | | | |
| | Peptide (Guard Column) | | | |
| Injection volume | 20 μL | | | |
| Detector | Radiometric Gabi Star - UV (220 nm) | | | |
| Pump program | Time (min) | A (%) | B (%) | Curve |
| | 0-1 | 90 | 10 | 0 |
| | 1-16 | 70 | 30 | 1 |
| | 16-18 | 70 | 30 | 0 |
| | 18-19 | 40 | 60 | 1 |
| | 19-21 | 40 | 60 | 0 |
| | 21-21.5 | 90 | 10 | 1 |
| | 21.5-24 | 90 | 10 | 0 |

| | |
|---|---|
| Retention time | free ⁶⁸Ga: ~1.5 |
| (min) | |
| | ⁶⁸Ga-PSMA-R2: ~14.9 |
| Detection time | 22.5 min |
| Run time | 24 min |

### Example 1: Development of a Method for Radiolabeling PSMA-R2 with 68Gallium Using a Two-Vial Kit

### 1. Description and Composition of the 2-Vial Kit

The applicant developed a sterile 2-vial kit which consists of:
Vial 1: PSMA-R2, 30 μg, powder for solution for injection, to be reconstituted with a solution of gallium-68 chloride (⁶⁸GaCl3) in HCl eluted from a ⁶⁸Ge/⁶⁸Ga generator;
Vial 2: Reaction buffer. Vial 2 is to be added to the reconstituted Vial 1.

The kit is used in combination with a solution of ⁶⁸Ga in dilute HCl eluted from a ⁶⁸Ge/⁶⁸Ga generator to prepare ⁶⁸Ga-PSMA-R2 as radiolabelled imaging product for intravenous injection.

The volume of ⁶⁸Ga-PSMA-R2 solution for injection, corresponding to the radioactive dose to be administered, is calculated according to the estimated time of injection, on the basis of the current activity provided by the generator and of physical decay of the radionuclide (half-life=68 min).

Vial 1 is a powder for solution for injection containing 30 μg PSMA-R2 as active ingredient, packed in 10 mL Ultra inert Type I Plus glass vials.

The composition of Vial 1 is provided in Table 1.

TABLE 1
Composition of Vial 1 powder for solution for injection

| | Composition | | Quality |
|---|---|---|---|
| Component | (per vial) | Function | Standard |
| PSMA-R2 | 30 μg | Active substance | In-house |
| D-Mannitol | 20 mg | Bulking agent | Ph. Eur/USP* |
| Water for injection ** | Qs | Solvent | Ph. Eur/USP* |
| Nitrogen | Qs | Inert blanket | Ph. Eur/USP* |

*current version
** Water for injection is eliminated during the lyophilisation process

The composition of Vial 2 is provided in Table 2.

TABLE 2
Composition of Vial 2 powder for solution for injection

| | Quality Standard | | Quantity |
|---|---|---|---|
| Component | (and Grade, if applicable) | Purpose | per vial |
| Formic acid | In-house | pH adjuster | 60 mg |
| Sodium hydroxide | In-house | pH adjuster | 56.5 mg |
| Water for injection | Ph. Eur/USP* | Solvent | qs |

### 2. Powder Vial (Vial 1)

As described above, Vial 1 (PSMA-R2, 30 μg, powder for solution for injection) is part of a radiopharmaceutical kit which also contain a reaction buffer (Vial 2) and an accessory cartridge.

The kit has to be used in combination with a solution of ⁶⁸Ga in HCl provided by a ⁶⁸Ge/⁶⁸Ga generator to obtain ⁶⁸Ga-PSMA-R2 solution for injection, being the Radiolabelled Imaging Product, which can be directly injected to the patient.

### 2.1 Components of the Drug Product

The drug product contains PSMA-R2 as active ingredient and mannitol as excipient.

### 2.1.1 Drug Substance

The active substance is the PSMA-R2 peptide, a 7-meraminoacid sequence covalently bound to a chelator (DOTA) through the C6 (6-aminohexanoic acid) linker. It is the compound of formula (II).

The sequence of PSMA-R2 is: HO-Glu-CO-Lys(Ne-4Bromobenzyl-Ne′-Ahx-DOTA)-OH, molecular formula: C41H63BrN8O15.

### 2.1.2 Excipients

The excipients chosen for the composition of Vial 1 are added to maintain stability of the active substance in the final formulation, to assure safety and efficacy of the drug product and also to obtain the required radiochemical purity of the ⁶⁸Ga-PSMA-R2 solution during the reconstitution procedure. The excipients selected lead to a drug product with the required pharmaco-technical characteristics.

A brief description of each excipient is provided as follows:

### Mannitol

Mannitol is used as bulking agent. Since peptide drugs are very potent, very small quantities are required in the drug product. In the absence of a bulking agent, the product processing becomes not suitable from technological point of view. Bulking agents allow pharmaceutical processing and the production of a presentable lyophilisate product.

### 2.2 Drug Product

### 2.2.1 Formulation Development

The formulation development has been performed with the aim of identifying the reaction mixture composition able to allow a simple labelling of the DOTA-molecule based on direct reconstitution with the eluate from commercially available ⁶⁸Ge/⁶⁸Ga generators without any processing of the eluate or any additional purification step.

The goal of this project was to develop the PSMA-R2 small molecule to be used as radiotracer for the detection of prostate tumors.

Vial 1 is a lyophilisate powder containing the peptide as active ingredient which is radiolabeled with ⁶⁸Ga during the radiolabelling procedure.

Initial efforts to develop a suitable formulation for PSMA-R2 (Vial 1) have involved tests in liquid form.

The drug product manufacturer focused the development work on the selection of the appropriate excipients in relation with the PSMA-R2 characteristics in order to obtain a finished product meeting the specifications commonly required for radiopharmaceutical preparations
⁶⁸Ga-PSMA-R2 (HPLC): >92%
Free ⁶⁸Ga³⁺ (HPLC): <2%
Non-complexed ⁶⁸Ga³⁺ species (ITLC): <3%

The development work including the relevant performed studies is described starting from the selection of the active ingredient amount and appropriate excipients.

### 2.2.1.1 Selection of the PSMA-R2 Amount

Increasing amounts of PSMA-R2 were tested using the Galliapharm, E&Z ⁶⁸Ge/⁶⁸Ga generator (1850 MBq) with the aim of identifying the minimum amount necessary to obtain a radiochemical purity ≥92% and the free ⁶⁸Ga<2% (as determined by HPLC analysis).

The following HPLC analyses, summarized in Table 3, show that the labelling performed with 5 μg of PSMA-R2 do not meet the specification (⁶⁸Ga free %<2%). The labelling carried out with 10 μg shows that the ⁶⁸Ga free % value is very close to the specification limit. The results clearly improve with the amount of PSMA-R2 above 15 μg.

The biodistribution studies carried out on the ⁶⁸GaPSMA-R2 molecule with the current specific activity indicate a favourable biodistribution profile in tumor models (major uptake in tumor and kidneys, relatively low uptake in other organs). The in vivo biodistribution data don't indicate a particular need for increasing the cold peptide in the formulation.

Therefore, based on all these considerations, 30 μg was set as the final amount as it satisfies our radiolabelling, stability and biodistribution requirements.

TABLE 3
PSMA-R2 amount - effect of the amount of PSMA-R2 on RCP %

| | HPLC | | | |
|---|---|---|---|---|
| | ⁶⁸GaPSMA- | ⁶⁸GaPSMA- | | |
| | R2 | R2 | | ⁶⁸Ga free |
| PSMA-R2 | Test results | Acceptance | ⁶⁸Ga free | Acceptance |
| (μg) | (%) | criterion | Test results (%) | criterion |
| 5 | 94.82 | 292% | 3.10* | ≤2% |
| 10 | 96.05 | 292% | 1.90 | ≤2% |
| 15 | 97.12 | 292% | 1.06 | ≤2% |
| 30 | 98.35 | 292% | 0.57 | ≤2% |

*The result is out of specification

Our development study was also focused on the selection of potential antioxidant agent and bulking agent. The radiolabelling procedure has also been thoroughly evaluated.

### 2.2.1.2 Selection of the Critical Excipients

### Selection of the Antioxidant Agent

The presence of a radical scavenger; with its antioxidant properties allow protecting PSMA-R2 from the radiolysis.

The attention focused on gentisic acid. Tests were made in order to identify the lowest amount of the antioxidant agent able to exert the desired protective function, without interfering with the labelling.

The labelling has been tested, varying the amount of antioxidant agents keeping constant other parameters, primarily to identify the concentration that not hampering the ⁶⁸Ga-incorporation into the DOTA-molecule.

The molecule was labelled with ⁶⁸Ga using E&Z generator with an activity in the range of 2030 mCi. The labelling was performed at 95° C. for 7 minutes with Gallium buffer (pH 3.2-3.8). Experiments were performed by testing different amount of gentisic acid as radiolytic scavenger and peptide amounts (15 μg and 30 μg). In all tests 20 mg of mannitol were added as cake forming.

In the table below are summarized the labelling conditions and the results obtained.

As demonstrated by the results shown in the Table 4, the radiochemical purity of ⁶⁸GaPSMA-R2 is always higher than 92% up to 4 hours of stability, already without the gentisic acid. Furthermore, the ⁶⁸Ga free is always under 2%, when gentisic acid is not used in the formulation. These preliminary results indicate that the molecule shows a good stability to the radiolytic degradation.

The following tests carried out by increasing the amount of gentisic acid do not seem to show an improvement in the stability of the molecule. The maximum amount of gentisic acid which gives good radiochemical results is 2 mg, while using 5 mg of gentisic acid the results do not meet the specifications. This is probably due to the partially competition for the ⁶⁸Ga complexation that occurs between the DOTA chelator and the gentisic acid, which presents a chelating functional group suitable for the metal ions complexation (carboxylic group). The influence of the gentisic acid becomes evident only at high amount (5 mg) since it is a much weaker chelating agent than the DOTA molecule.

Therefore, based on these experimental results it was concluded that an antioxidant agent is not needed in the drug product composition.

TABLE 4
Gentisic acid amount: effect on ⁶⁸GaPSMA-R2 stability

| | | | | ⁶⁸Ga free | ⁶⁸GaPSMA-R2 |
|---|---|---|---|---|---|
| | | | | HPLC (%) | HPLC (%) |
| | | Gentisic acid | Activity | Target | Target |
| PSMA-R2 (μg) | Mannitol (mg) | (mg) | mCi, MBq | value: ≤2% | value: ≥92% |
| 15 | 20 | — | 24.15 mCi | t0h 1.44 | t0h 96.30 |
| | | | 893.6 MBq | t2h 1.74 | t2h 95.61 |
| | | | | t4h 1.82 | t4h 95.10 |
| 30 | 20 | — | 30.13 mCi | t0h 0.57 | t0h 98.35 |
| | | | 1114.8 MBq | t2h 0.67 | t2h 97.75 |
| | | | | t4h 0.72 | t4h 97.20 |
| 30 | 20 | — | 29.28 mCi | t0h 0.74 | t0h 96.85 |
| | | | 1083.4 MBq | t2h 0.77 | t2h 95.34 |
| | | | | t4h 1.15 | t4h 95.11 |
| 30 | 20 | — | 20.28 mCi | t0h 1.40 | t0h 97.47 |
| | | | 750.4 MBq | t2h 1.43 | t2h 97.40 |
| | | | | t4h 1.44 | t4h 97.24 |
| 30 | 20 | 0.006 | 29.25 mCi | t0h 0.64 | t0h 97.77 |
| | | | 1083.3 MBq | t2h 0.75 | t2h 97.44 |
| | | | | t4h 0.97 | t4h 96.33 |
| 30 | 20 | 0.020 | 29.44 mCi | t0h 0.90 | t0h 95.87 |
| | | | 1089.3 MBq | t2h 1.13 | t2h 95.28 |
| | | | | t4h 1.39 | t4h 95.06 |
| 30 | 20 | 0.100 | 28.89 mCi | t0h 0.74 | t0h 96.38 |
| | | | 1068.9 MBq | t4h 0.98 | t4h 95.73 |
| 30 | 20 | 0.100 | 28.74 mCi | t0h 0.63 | t0h 96.00 |
| | | | 1063.4 MBq | t2h 0.84 | t2h 95.52 |
| | | | | t4h 1.24 | t4h 95.01 |
| 15 | 20 | 0.200 | 23.61 mCi | t0h 1.33 | t0h 96.36 |
| | | | 873.6 MBq | t2h 1.44 | t2h 95.93 |
| | | | | t4h 1.79 | t4h 95.63 |
| 30 | 20 | 0.200 | 29.92 mCi | t0h 0.61 | t0h 96.34 |
| | | | 1107.0 MBq | t2h 0.65 | t2h 95.62 |
| | | | | t4h 1.10 | t4h 95.02 |
| 30 | 20 | 1.000 | 28.73 mCi | t0h 0.88 | t0h 95.84 |
| | | | 1063.0 MBq | | |
| 30 | 20 | 2.000 | 25.50 mCi | t0h 1.64 | t0h 93.42 |
| | | | 943.5 MBq | | |
| 30 | 20 | 5.000 | 24.51 mCi | t0h 2.76* | t0h 91.59* |
| | | | 906.9 MBq | | |

*The result is out of specifications.

### Selection of the Bulking Agent

The formulation was finally completed with the addition of a bulking agent for the freeze-drying process.

Among the bulking agent usually proposed for the lyophilisation of peptides, mannitol has been selected as it produces a cake with good characteristics in terms of aspect, stability and moisture in freeze-drying processes

Radiolabelling tests have been performed with E&Z generator (activity 30 mCi-1110 MBq) on two different formulations by changing the amount of mannitol, without using the gentisic acid as described in the table below. Both formulations do not negatively affect the radiolabelling results, however the results obtained on the formulation with 20 mg mannitol recorded better results. The amount of the mannitol selected was 20 mg. Moreover, mannitol is described in literature as good scavengers of OH radicals.

TABLE 5
Different bulking agent amount

| | | | ⁶⁸Ga free | ⁶⁸GaPSMA- |
|---|---|---|---|---|
| | | | HPLC | R2 HPLC |
| PSMA-R2 | Mannitol | Activity | Target | Target |
| (μg) | (mg) | mCi, MBq | value: ≤2% | value: ≥92% |
| 30 | 20 | 30.13 mCi | t0h 0.57 | t0h 98.35 |
| | | 1114.8 MBq | t2h 0.67 | t2h 97.75 |
| | | | t4h 0.72 | t4h 97.20 |
| 30 | 40 | 20.83 mCi | t0h 0.89 | t0h 97.34 |
| | | 770.71 MBq | t2h 1.00 | t2h 97.05 |
| | | | t4h 1.31 | t4h 96.60 |

### 2.3 Effect of the pH on the Radiochemical Purity

The aim of these tests was to evaluate the impact of the labelling pH on the radiochemical purity of different formulations. The pH plays an important role not only on the coordination chemistry, but also on the stability of peptides and small molecules in liquid formulations. Regarding the chemistry of the ⁶⁸Ga, the pH changes influence the labelling behaviour dramatically:

Owing to the aqueous chemistry of gallium-68, the pH value has to be kept low to avoid the formation of ⁶⁸Ga oxide and hydroxide species. On the other hand, the pH value has to be high enough to deprotonate a sufficient number of donor functions of the chelator.

The specification of the pH value defined for these ⁶⁸Ga-labelled products is between 3.2-3.8. This range of pH covers the values that are compatibles for the complexation of ⁶⁸GaCl3 with the DOTA chelator by using our labelling approach.

Based on these considerations, three different formulations have been labelled with E&Z generator at different pH (3.0, 3.2, 3.8, 4.0) by changing the volume of the gallium buffer (Vial 2).

The following formulations have been selected on the basis of the best radiochemical purity results obtained with the lowest amount of antioxidant agent (see Table 4).
Formulation 1: 30 μg PSMA-R2, 20 mg mannitol;
Formulation 2: 30 μg PSMA-R2, 6.0 μg gentisic acid, 20 mg mannitol;
Formulation 3: 30 μg PSMA-R2, 100 μg gentisic acid, 20 mg mannitol;

As can be noted in the table below, all the radiolabelling carried out on the formulation 1 with a final pH between 2.90-3.35 show results which are within the specifications.

TABLE 6
Formulation 1 labelled at lower pH

| | | | iTLC | | |
|---|---|---|---|---|---|
| | | | ⁶⁸Ga non | | HPLC |
| | | | complexed | HPLC | ⁶⁸GaPSMA- |
| | | | species (%) | ⁶⁸Ga free (%) | R2 (%) |
| | | | Acceptance | Acceptance | Acceptance |
| Formulation 1 | Amount | pH | criterion: ≤3% | criterion: ≤2% | criterion: ≥92% |
| Mannitol | 20 mg | 2.90 | 0.56 | 1.11 | 96.19 |
| PSMA-R2 | 30 μg | 3.18 | 0.95 | 1.01 | 95.98 |
| | | 3.35 | 0.40 | 0.77 | 96.89 |

The Table 7 shows the results carried out on the formulation 1 at pH>3.80: all the results meet the specifications.

TABLE 7
Formulation 1 labelled at higher pH

| | | | iTLC | | |
|---|---|---|---|---|---|
| | | | ⁶⁸Ga non | | HPLC |
| | | | complexed | HPLC | ⁶⁸GaPSMA- |
| | | | species (%) | ⁶⁸Ga free(%) | R2(%) |
| | | | Acceptance | Acceptance | Acceptance |
| Formulation 1 | Amount | pH | criterion: ≤3% | criterion: ≤2% | criterion: ≥92% |
| Mannitol | 20 mg | 3.90 | t0h 0.84 | t0h 1.48 | t0h 96.87 |
| | | | | t2h 1.72 | t2h 96.09 |
| PSMA-R2 | 30 μg | | t4h 2.12 | t4h 1.61 | t4h 96.21 |

As can be noted in the Table 8, all the radiolabelling tests carried out on the formulation 2 with a final pH<3.40 show results that are within the specifications.

TABLE 8
Formulation 2 labelled at lower pH

| | | | iTLC | | |
|---|---|---|---|---|---|
| | | | ⁶⁸Ga non | | HPLC |
| | | | complexed | HPLC | ⁶⁸GaPSMA- |
| | | | species (%) | ⁶⁸Ga free (%) | R2 (%) |
| | | | Acceptance | Acceptance | Acceptance |
| Formulation 2 | Amount | pH | criterion: ≤3% | criterion: ≤2% | criterion: ≥92% |
| Mannitol | 20 mg | 3.10 | 0.25 | 0.78 | 97.35 |
| PSMA-R2 | 30 μg | 3.27 | 0.79 | 1.07 | 96.09 |
| Gentisic acid | 6.0 μg | 3.40 | 0.94 | 1.24 | 94.90 |

The Table 9 shows the results carried out on the formulation 2 at pH>3.80: all the results meet the specifications.

TABLE 9
Formulation 2 labelled at higher pH

| | | | ITLC | | |
|---|---|---|---|---|---|
| | | | ⁶⁸Ga non | | HPLC |
| | | | complexed | HPLC | ⁶⁸GaPSMA- |
| | | | species (%) | ⁶⁸Ga free (%) | R2 (%) |
| | | | Acceptance | Acceptance | Acceptance |
| Formulation 2 | Amount | pH | criterion: ≤3% | criterion: ≤2% | criterion: ≥92% |
| Mannitol | 20 mg | 3.96 | 0.40 | 0.86 | 96.54 |
| PSMA-R2 | 30 μg | | | | |
| Gentisic acid | 6.0 μg | | | | |

The Table 10 shows the results carried out on the formulation 3 at pH<3.2: all the results do not meet the specification (RCP %<92%)

TABLE 10
Formulation 3 labelled at lower pH

| | | | iTLC | | |
|---|---|---|---|---|---|
| | | | ⁶⁸Ga non | | HPLC |
| | | | complexed | HPLC | ⁶⁸GaPSMA- |
| | | | species (%) | ⁶⁸Ga free (%) | R2 (%) |
| | | | Acceptance | Acceptance | Acceptance |
| Formulation 3 | Amount | pH | criterion: ≤3% | criterion: ≤2% | criterion: ≥92% |
| Mannitol | 20 mg | 3.12 | 0.89 | 1.47 | 87.45* |
| PSMA-R2 | 30 μg | 3.21 | 0.90 | 1.20 | 90.57* |
| Gentisic acid | 0.1 mg | 3.20 | 0.74 | 0.81 | 91.57* |
| | | 2.96 | 1.26 | 1.32 | 85.81* |

*The result is out of specifications

The Table 11 shows the results carried out on the formulation 3 at pH >3.80: all the results meet the specifications.

TABLE 11
Formulation 3 labelled at higher pH

| | | | iTLC | | |
|---|---|---|---|---|---|
| | | | ⁶⁸Ga non | | HPLC |
| | | | complexed | HPLC | ⁶⁸GaPSMA-R2 |
| | | | species (%) | ⁶⁸Ga free (%) | (%) |
| | | | Acceptance | Acceptance | Acceptance |
| Formulation 3 | Amount | pH | criterion: ≤3% | criterion: ≤2% | criterion: ≥92% |
| Mannitol | 20 mg | 3.80 | 0.44 | 0.61 | 96.32 |
| PSMA-R2 | 30 μg | 4.00 | 0.15 | 0.69 | 96.91 |
| Gentisic acid | 0.1 mg | | | | |

In conclusion, the results collected show a significant decrease of radiochemical purity of ⁶⁸GaPSMA-R2 in the formulation 3 (gentisic acid amount 100 μg) only when the final pH of the labelling is lower, between 3.0-3.2. The same formulation, tested at a final pH around the upper limit (pH about 3.8) show always results within the specifications.

The tests carried out on the formulation 1 (no gentisic acid) and on the formulation 2 (gentisic acid amount 6 μg) show always results within the specification either at lower pH (3.0-3.2) that at higher pH (3.8-4.0).

On the basis of these considerations, it can be assumed that there is a negative influence of the gentisic acid only when the final pH of labelling is lower, around 3.2. The HPLC results demonstrate that these specific conditions lead to an increase of the radioactive impurities in the radiolabelled product.

For these reasons, we have tested some formulations with different amount of gentisic acid, always keeping the final labelling pH around the lower limit value (pH 3.2). Our aim was to understand more clearly if the gentisic acid could adversely affect the radiochemical purity of ⁶⁸GaPSMA-R2.

The radiolabelling results collected in Table 12 demonstrate that, when the final labelling pH is around 3.2, the amount of 200 μg of gentisic acid in the formulation could adversely affect the RCP % of the product. The results obtained with 100 μg are slightly above the specifications while, further lowering the amount below 12 μg, the results clearly improve. Based on all these results, it can be concluded that the presence of gentisic acid has a negative impact on the radiochemical purity of the radiolabelled solution, promoting the appearance of potential impurities (other radioactive species) in a low pH solution.

TABLE 12
⁶⁸GaPSMA-R2 labelling results obtained at pH 3.0-3.2

| | Gentisic | | | HPLC | HPLC |
|---|---|---|---|---|---|
| PSMA-R2 | acid | Activity | Labelling | ⁶⁸Ga | ⁶⁸GaPSMA- |
| (μg) | (mg) | (mCi) | pH | free (%) | R2 (%) |
| 30 | 0.200 | 20.13 | 3.22 | t0h 1.65 | t0h: 85.46* |
| 30 | 0.100 | 16.21 | 3.20 | t0h 0.90 | t0h 92.29 |
| 30 | 0.020 | 17.55 | 3.15 | t0h 1.32 | t0h 95.10 |
| 30 | 0.012 | 29.19 | 3.11 | t0h 0.99 | t0h 93.94 |
| | | | | t2h 1.50 | t2h 93.34 |
| | | | | t4h 2.51* | t4h 91.65* |
| 30 | 0.010 | 14.25 | 3.21 | t0h 0.94 | t0h 97.01 |
| 30 | 0.006 | 10.09 | 3.18 | t0h 0.81 | t0h 96.85 |
| | | | | t2h 1.15 | t2h 95.37 |
| | | | | t4h 1.21 | t4h 94.40 |
| 30 | 0.006 | 26.23 | 3.20 | t0h 1.06 | t0h 95.46 |
| 30 | 0.006 | 27.65 | 3.25 | t0h 0.97 | t0h 97.10 |
| | | | | t2h 1.01 | t2h 97.00 |
| | | | | t4h 1.13 | t4h 97.08 |
| 30 | — | 26.98 | 3.25 | t0h 0.82 | t0h 97.59 |
| 30 | — | 27.56 | 3.23 | t0h 1.62 | t0h 96.76 |
| | | | | t2h 1.59 | t2h 96.26 |
| | | | | t4h 1.63 | t4h 96.64 |
| 30 | — | 27.86 | 3.20 | t0h 1.04 | t0h 96.90 |
| | | | | t2h 1.41 | t2h 96.41 |

*The result is out of specifications

### 2.4 Radiolabelling Procedure

Based on the 2-vials kit design, a 3-step labelling procedure has been developed as follows:
1. direct reconstitution of the powder vial with the solution of ⁶⁸Ga in HCl provided by the ⁶⁸Ge/⁶⁸Ga E&Z generator.
2. addition of the necessary volume of reaction buffer
3. heating at 95° C. for at least 7 minutes (do not exceed 10 minutes heating)

At this point the ⁶⁸Ga-PSMA-R2 solution is ready for administration.

During the labelling procedure development, different time and temperature conditions have been tested.

The dependence of the labelling efficiency on the temperature has been studied to identify a value giving a good incorporation in a timeframe compatible with the short half-life of the ⁶⁸Ga (68 minutes).

The incorporation of the ⁶⁸Ga into the DOTA chelating moiety, is known to require heating to be accomplished.

The testing started with the elution of the generator and the addition of reaction buffer at room temperature, following heating at 95° C. The results are summarized in the table below.

TABLE 13
Elution and buffer adding at RT

| PSMA-R2 | Temperature | HPLC | HPLC |
|---|---|---|---|
| (μg) | (° C.) | ⁶⁸GaCl₃ (%) | ⁶⁸GaPSMA-R2 (%) |
| 30 | 95 | t0h 0.57 | t0h 98.35 |
| 15 | 95 | t0h 1.37 | t0h 96.58 |

Also the labelling at 70° C., 80° C., 90° C., 95° C. has been tested with different reaction times (3, 5 and 7 minutes) and 100° C. The ⁶⁸Ga-radiolabelling performed at 70° C. for 7 minutes showed inadequate ⁶⁸Ga incorporation. The increase of the temperature to 80° C., promoted the incorporation above 94%. At 95° C., the incorporation is almost completed after 5 minutes. Based on these observations, 95° C. for 7 minutes showed to be the most conservative labelling condition, able to guarantee incorporation above 95% without significant fragmentation, even in the case of oscillation of the temperature in the range of ±5° C.

TABLE 14
Labelling at different temperatures and times

| | | | | | iTLC |
|---|---|---|---|---|---|
| Labelling | | | | | ⁶⁸Ga non |
| temperature | PSMA-R2 | Labelling | HPLC | HPLC | complexed |
| (° C.) | (μg) | time (min) | ⁶⁸GaCl₃ (%) | ⁶⁸GaPSMA-R2 (%) | species |
| 70 | 30 | 7 | 6.81* | 86.23* | — |
| 80 | 30 | 7 | 1.03 | 94.97 | — |
| 90 | 30 | 7 | 1.07 | 94.92 | 2.08 |
| 95 | 15 | 3 | 15.80* | 82.21* | — |
| 95 | 15 | 5 | 3.00* | 94.26 | — |
| 95 | 15 | 7 | 1.87 | 95.05 | — |
| 95 | 30 | 3 | 14.7* | 83.23* | 20.41* |
| 95 | 30 | 5 | 1.66 | 93.39 | 2.32 |
| 95 | 30 | 7 | 0.74 | 96.38 | 0.33 |
| 100 | 30 | 7 | 0.92 | 95.75 | 0.31 |

*The result is out of specifications

Tests were performed also in order to evaluate the admissible delay between the addition of the ⁶⁸Ga eluate and the addition of the buffer still providing a Radiolabeled Imaging Product meeting the specifications.

The reconstitution procedure was tested by waiting after the reconstitution of the lyophilized formulation and before the addition of the buffer, an increasing number of minutes. The radiochemical purity was tested by HPLC.

The results showed that a delay in the buffer addition up to 15 minutes does not affect the success of the labelling.

TABLE 15
Test on buffer addition delay - effect on stability/purity

| | HPLC | HPLC |
|---|---|---|
| Test | ⁶⁸GaCl₃ (%) | ⁶⁸GaPSMA-R2 (%) |
| 2 minutes delay | 0.57 | 98.35 |
| 5 minutes delay | 1.10 | 96.81 |
| 10 minutes delay | 1.68 | 95.22 |
| 15 minutes delay | 1.27 | 95.05 |

The possibility of adding the Gallium buffer to the Vial 1 before the elution of the ⁶⁸Ge/⁶⁸Ga generator has also been tested. Following this procedure, in Table 19 are reported the labelling results obtained by HPLC analysis.

TABLE 16
Addition of the buffer before the elution:
radiochemical purity evaluation

| PSMA-R2 | HPLC | HPLC |
|---|---|---|
| (μg) | ⁶⁸GaCl₃ | ⁶⁸GaPSMA-R2 |
| 15 | 4.15* | 92.96 |
| 30 | 5.49* | 91.45* |

*The result is out of specifications

The radiolabelling tests performed with the addition of the gallium buffer before the elution step leads to results that do not meet the specifications, therefore this option is discarded.

### Radiolabelling Procedure with ⁶⁴Cu

As well as for ⁶⁸Ga, based on the 2-vials kit design, a ⁶⁴Cu labelling procedure has been developed as follows:
1. direct reconstitution of the powder vial with the solution of ⁶⁴Cu in HCl provided by the ⁶⁴Cu produced via cyclotron.
2. addition of the necessary volume of reaction buffer
3. heating at 70° C. for at least 15 minutes

During the labelling procedure development, different time and temperature conditions have been tested.

The dependence of the labelling efficiency on the temperature has been studied to identify a value giving a good incorporation and a good stability up to 24 h without incurring degradation of the product.

The testing started with the incorporation at room temperature. Table 17 reports the results achieved using PSMA R2 peptide and PSMA R2 kit incubated at RT.

TABLE 17
Labelling at RT

| | | | | HPLC | HPLC |
|---|---|---|---|---|---|
| Test | PSMA-R2 | Temperature | Time | ⁶⁴CuCl₂ | ⁶⁴CuPSMA-R2 |
| item | (μg) | (° C.) | (min) | (%) | (%) |
| peptide | 35 | RT | 50 | t0h 0.0 | t0h 100 |
| kit | 30 | RT | 30 | t0h 2.6 | t0h 92.6 |

Labelling conditions at 40° C., 70° C. and 95° C. have been tested using different reaction times and pH as reported in table 18.

TABLE 18
Labelling at different temperatures, times and pH

| | | | | | | iTLC | ITLC |
|---|---|---|---|---|---|---|---|
| | | | | | | ⁶⁸Ga non | ⁶⁸Ga non |
| | | | | HPLC | HPLC | complexed | complexed |
| Labelling | Labelling | | HPLC | ⁶⁴CuPSMA-R2 | ⁶⁴CuPSMA-R2 | species | species |
| temperature | time | | ⁶⁴CuCl₂ | (%) | (%) | (%) | (%) |
| (° C.) | (min) | pH | (%) | t0 | t24 | t0 | t24 |
| RT | 90 | 3.1 | 0 | 91.87 | 81.09 | n.d | n.d |
| RT | 60 | 3.5 | 0 | 48.3* | 91.82 | 0.4 | 0.4 |
| RT | 60 | 3.1 | 0 | 81.6** | 90.48 | 2.5 | 4.8 |
| RT | 30 | 3.9 | 0 | 94.6 | 94.59 | 0.3 | 0.5 |
| 40 | 20 | 3.1 | 0 | 91.24 | 84.99 | n.d- | n.d |
| 95 | 7 | 3.1 | 0 | 88.62*** | 92.88 | 4.2- | 5.7*** |
| 95 | 7 | 3.9 | 0 | 94.52 | 93.75 | 0.5 | 0.8 |
| 70 | 15 | 3.9 | 0 | 95.1 | 96.12 | 0.3 | 0.7 |
| 70 | 15 | 3.9 | 0 | 94.9 | 95.13 | 0.4 | 0.4 |
| 70 | 15 | 4.8 | 0 | 94.85 | 94.59 | 0.5 | 0.7 |
| 70 | 15 | 3.5 | 0 | 96.09 | 96.25 | 0.3 | 1.2 |
| 70 | 15 | 4.0 | 0 | 94.7 | 93.69 | 4.2 | 1.6 |
| 70 | 15 | 4.5 | 0 | 95.6 | 95.6 | 0.4 | 1.1 |

*The result is affected by analytical issue.
**Reaction not completed
***Result out of specification

The ⁶⁴Cu-radiolabelling performed at RT showed inadequate ⁶⁴Cu incorporation when pH is lower than 4. The increase of the temperature to 70° C., promoted the incorporation above 94%. At 95° C., the incorporation is good after 7 minutes. Based on these observations, 70° C. for 15 minutes showed to be the most conservative labelling condition, able to guarantee incorporation above 94% without significant fragmentation up to 24 h.

Moreover, the last three results, obtained using 70° C. for 15 minutes as heating step, showed that good results can be achieved by using different radioconcentration from 100 MBq/mL till 200 MBq/mL in a final volume ranging from 3 to 8 mL.

These results demonstrate that the ⁶⁴CuPSMA-R2 can be obtained mimicking the same range used for ⁶⁸Ga radiolabelling concentration considering that a highly charged ⁶⁸Ge/⁶⁸Ga generator can elute about 200 MBq/mL.

### 2.5 Final Formulation and Detailed Composition

Based on all development performed on the formulation as above presented, the final chosen formulation of vial 1 is the following:

TABLE 19
Final formulation

| | Quality Standard (and | | Quantity |
|---|---|---|---|
| Component | Grade, if applicable) | Purpose | per vial |
| AAA-PSMA-R2 | In-house | Active | 30 μg |
| D-Mannitol | Ph. Eur/USP* | Bulking agent | 20 mg |
| Water for injection | Ph. Eur/USP* | Solvent | Qs |
| Nitrogen | Ph. Eur/USP* | Inert blanket | Qs |

*current version
**Water for injection is eliminated during the lyophilisation process

The radiolabelled formulation is the following:

TABLE 20
Final radiolabelled formulation

| Component | 5 mL of HCl 0.1N | |
|---|---|---|
| PSMA-R2 content | 30 | μg |
| ⁶⁸Ga-PSMA-R2 content | ≤0.0161 | μg |
| Total radioactivity | ≤1110 | MBq |
| Specific Activity (GBq/total peptide) | ≤36.5 GBq/μ | mol |
| Radioconcentration | ≤202 | MBq/mL |
| Volume | ≤5.5 | mL |

| Content of Excipients | mg/vial |
|---|---|
| Mannitol | 20 |
| Formic Acid | 30 |
| Sodium Hydroxide | 28.25 |
| Hydrochloric acid | 18.22 |
| Water for Injection | Add ≤5.5 ml |

As demonstrated during the development process of the product, the radiochemical purity of ⁶⁸Ga-PSMA-R2 is always highly over 92% up to 4 hours even without the gentisic acid (Table 4). This behaviour indicate that the molecule presents an intrinsic stability to the radiolytic degradation.

The addition of the gentisic acid in the formulation does not seem to show an improving effect on the stability of the ⁶⁸GaPSMA-R2 product, therefore this excipient was not included in the final formulation.

### 2.6 Evaluation of Specification

The final formulation has been tested in order to confirm the results obtained during the development.

TABLE 21
Labelling results obtained with the final formulation

| | | | iTLC | | |
|---|---|---|---|---|---|
| | | | ⁶⁸Ga not | | HPLC |
| Final | Activity | | complexed | HPLC ⁶⁸Ga | ⁶⁸GaPSMA- |
| Formulation | mCi, MBq | pH | species | free (%) | R2 (%) |
| Mannitol | 29.28 mCi | 3.59 | t0h 0.20 | t0h 0.74 | t0h 96.85 |
| PSMA-R2 | 1083.4 | | | t2h 0.77 | t2h 95.34 |
| | MBq | | | t4h 1.15 | t4h 95.11 |
| Mannitol | 30.13 mCi | 3.70 | t0h 0.32 | t0h 0.57 | t0h 98.35 |
| PSMA-R2 | 1114.8 | | | t2h 0.67 | t2h 97.75 |
| | MBq | | | t4h 0.72 | t4h 97.20 |
| Mannitol | 20.28 mCi | 3.63 | t0h 0.86 | t0h 1.40 | t0h 97.47 |
| PSMA-R2 | 750.4 | | | t2h 1.43 | t2h 97.40 |
| | MBq | | | t4h 1.44 | t4h 97.24 |

Liquid formulations were performed targeting very high radiochemical purity values. This approach was followed to guarantee wide margins for the evolution from an R&D liquid formulation to a GMP lyophilized product still assuring adequate quality.

The free ⁶⁸Ga content was monitored by HPLC during development and the selected formulation demonstrated results consistently within the target limit. Based on this, and considering that the non-complexed ⁶⁸Ga species evaluated by ITLC include both colloidal and free ⁶⁸Ga, continuous monitoring of the latter parameter is not considered as necessary.

In the passage from results obtained in-house during development to a GMP product to be locally reconstituted, it was also considered adequate to set the specification for the non-complexed ⁶⁸Ga species by ITLC<5%. This specification guarantees an incorporation of the radioisotope not lower than 95% which is in line with common requirements for kit-based radiopharmaceuticals.

The ⁶⁸Ga-PSMA-R2 radiochemical purity is checked by HPLC before release of the kit to assure that after reconstitution the ⁶⁸Ga-labelled peptide accounts for more than 90.0% of the total radioactivity when reconstitution instruction is properly applied.

In conclusion, based on the formulation development results and on the common requirements for radiopharmaceutical preparation, the following radiochemical specifications have been set for the release of the lyophilized GMP product at the manufacturing site:
⁶⁸Ga-PSMA-R2 (HPLC): ≥90.0%
Non-complexed ⁶⁸Ga³⁺ species (ITLC): <5.0%

### 3 Reaction Buffer Vial (Vial-2)

### 3.1 Formulation Development

### 3.1.1 Generalities

The formulation development of the Reaction buffer aimed to define a formulation that allows labelling of DOTA-molecule with high and reproducible complexation yields, by direct reconstitution with the eluate provided by the 68Ge/68Ga generator.

Such direct procedure makes the labelling process accessible and not dependent on the use of automatic synthesis modules, which are very expensive and available only in limited number of nuclear pharmacies.

The proposed reconstitution procedure does not require additional purification steps and provides a Radiolabelled Imaging Product which meets the pre-defined quality criteria.

This approach answers to an unmet need recognized in the nuclear medicine community.

It is commonly known that the main challenges in the development of the kit for radiopharmaceutical preparation are related to a successful labelling procedure, which, in the case of ⁶⁸Ga isotope, is limited by:
1 The difficulty of keeping constant a suitable pH value,
2 The competition of the metallic impurities in the complexation process.
3 Stability of the product and major indicator: radiochemical purity

These three aspects made direct application of the eluate coming from the ⁶⁸Ge/⁶⁸Ga generator in the labelling procedure impracticable so far.

By consequence, the first focus of the formulation development has been the research of a buffer capable to maintain with good reliability the desired pH value after recovering of the total eluate provided by the generator.

The pH value plays a key role in ⁶⁸Ga-labelling, since its changes influence the labelling behaviour dramatically:
Owing to the aqueous chemistry of gallium-68, the pH value has to be kept low to avoid the formation of ⁶⁸Ga oxide and hydroxide species.
On the other hand, the pH value has to be high enough to deprotonate a sufficient number of donor functions of the chelator.

Among the different available buffers, the first tested ones were already known and used for labelling with ⁶⁸Ga, such as HEPES (sulfonic acid derivative) or acetate buffer.

### 3.1.2 Buffer Selection

### HEPES

HEPES is not able to tolerate even low variation in the HCl solution volume and for, this reason, can be hardly applied to a kit designed for the direct reconstitution with the eluate coming from the ⁶⁸Ge/⁶⁸Ga generator whose volume cannot be strictly constant in the routine use.

Moreover, HEPES should not be left inside the injectable solution in such high concentration imposing a final purification after labelling which is not compatible with the kit approach.

TABLE 22
pH values obtained after mixing HEPES buffer
(500 μL of Na-HEPES 300 mg/mL) with HCl 0.1N

| | |
|---|---|
| Volume HCl | pH |

| 0.1N (mL) | Test 1 | Test 2 | Test 3 | Average pH |
|---|---|---|---|---|
| 4.2 | 6.34 | 6.28 | 6.33 | 6.32 |
| 4.4 | 5.80 | 5.50 | 5.69 | 5.66 |
| 4.6 | 4.51 | 4.51 | 4.53 | 4.52 |
| 4.8 | 4.03 | 4.03 | 4.03 | 4.03 |
| 5.0 | 3.78 | 3.78 | 3.78 | 3.78 |
| 5.2 | 3.61 | 3.61 | 3.60 | 3.60 |

### Acetate Buffer

Acetate buffer is also well known for the use in ⁶⁸Ga-labelling and provided quite stable pH values in preliminary tests. Nevertheless, it gave inconsistent results.

It is important to note that all the successful labelling documented in literature with HEPES and acetate arise from labelling procedure based on pre-processing steps of the eluate and final purification of the radiolabelled product.

### Alternative Buffers

Thereafter, the search for alternative buffers, compatible with injectable use, has focused on buffer whose pKa was in the range 3.2-4.2 thus assuring effective buffering ability at the optimal pH values for ⁶⁸Ga-labelling. In Table 23 are listed common organic acid with their pKa.

TABLE 23
pKa of common organic acid

| Buffer | pKa (20° C.) |
|---|---|
| Citric acid | 3.14 |
| Formic acid | 3.75 |
| Lactic acid | 3.86 |
| Succinic acid | 4.22 |

Citric acid was excluded for its ability to form stable complex with gallium. This is confirmed also by the existence of the well-known SPECT product ⁶⁷Ga-citrate.

Lactic acid proved as well to hamper the complexation of ⁶⁸Ga by the DOTA chelator providing more than 97% of free ⁶⁸Ga in a preliminary test of labelling.

Succinic acid was tested in the labelling with a 5 ml solution of ⁶⁸Ga in HCl 0.1 but, even establishing a reliable pH value around 3.4, never provided a satisfying final ⁶⁸Ga labelled DOTA-peptide, being the free ⁶⁸Ga content always higher than 8% in HPLC.

Finally, due to its pKa, formic acid was found to have a buffering capacity well centered at the pH value suitable for the ⁶⁸Ga complexation. Moreover, this buffer was deemed compatible with the intended pharmaceutical application since formic acid is classified as a class 3 (solvents with low toxic potential) residual solvent in the Pharmacopoeia and should have not been removed from the final injectable solution at the end of the labelling if kept below the permitted daily exposure (PDE).

Based on the Henderson-Hasselbalch equation which describes the behavior of the buffer systems, calculation were made on the amount of formic acid and of the alkaline counterpart necessary to have a final pH around 3.5 considering the contribute of the HCl coming from the generator.

Sodium hydroxide was selected as alkaline counterpart as it is a strong base able to compensate the strong HCl acid and to generate the conjugated base of formic acid needed to establish the buffer pair. An amount of formic acid of 30 mg with 28.25 mg of sodium hydroxide resulted adequate to keep the pH value around 3.5. Additionally, this formic acid amount is well below the PDE of 50 mg for Class 3 solvents.

The formate buffer with above concentration proved to be able to keep the pH in the range of 3.2-3.8 for a quite extended range of volume of HCl, not only after addition of the standard volumes of the eluates (5 mL of HCl 0.1 N and 4 mL of HCl 0.05 N thus mimicking the eluate characteristics of the most common commercially available ⁶⁸Ge/⁶⁸Ga generators). This ensure optimal labeling conditions, even in case of reduced eluate recovery from the generator, which is likely to happen in real practice, where strictly constant volumes cannot be assured.

As in a kit-type approach no pre-concentration of the generator eluate volume is foreseen, in order to avoid further dilution of the reaction mixture, the concentration of formic acid in the formulation was optimized to keep low the volume of the buffer needed for labelling. This is an advantage as the labeling at nanomolar peptide concentration requires small reaction volumes to maximize labeling yield.

Table 24 and Table 25 summarize the pH values measured after mixing suitable volumes of formate buffer with variable volumes of HCl 0.1 N, and 0.05 N.

TABLE 24
pH values obtained after mixing Formate buffer
(500 μL of formic acid 60 mg/mL, sodium
hydroxide 56.5 mg/mL) with 5 mL of HCl 0.1N

| | |
|---|---|
| Volume HCl | pH |

| 0.1N (mL) | Test 1 | Test 2 | Test 3 | Average pH |
|---|---|---|---|---|
| 3.6 | 3.70 | 3.69 | 3.73 | 3.70 |
| 3.8 | 3.62 | 3.58 | 3.60 | 3.60 |
| 4.0 | 3.54 | 3.50 | 3.50 | 3.51 |
| 4.2 | 3.47 | 3.42 | 3.44 | 3.44 |
| 4.4 | 3.42 | 3.36 | 3.39 | 3.39 |
| 4.6 | 3.40 | 3.31 | 3.33 | 3.35 |
| 4.8 | 3.34 | 3.28 | 3.27 | 3.30 |
| 5.0 | 3.28 | 3.22 | 3.21 | 3.24 |
| 5.2 | 3.24 | 3.18 | 3.17 | 3.20 |

TABLE 25
pH values obtained after mixing Formate buffer
(200 μL of formic acid 60 mg/mL, sodium
hydroxide 56.5 mg/mL) with 4 mL of HCl 0.05N

| Volume HCl | | | | |
|---|---|---|---|---|
| 0.05N (mL) | Test 1 pH | Test 2 pH | Test 3 pH | Average pH |
| 2.6 | 3.75 | 3.73 | 3.76 | 3.75 |
| 2.8 | 3.61 | 3.59 | 3.63 | 3.61 |
| 3.0 | 3.52 | 3.56 | 3.54 | 3.54 |
| 3.2 | 3.45 | 3.50 | 3.51 | 3.49 |
| 3.4 | 3.38 | 3.41 | 3.43 | 3.41 |
| 3.6 | 3.31 | 3.33 | 3.36 | 3.33 |
| 3.8 | 3.24 | 3.25 | 3.28 | 3.26 |
| 4.0 | 3.16 | 3.17 | 3.19 | 3.17 |

The adequacy of formate buffer was then confirmed by the success of the labelling demonstrating the absence of interfering effect on the ⁶⁸Ga chelation by the DOTA moiety. Globally formic acid/formate buffer in the above concentration proved:
To be able to compensate the acidity of the total eluate coming from the generator, without need of reduction or concentration of the eluate volume, making a kit-type direct labelling procedure feasible;
To guarantee a stable pH value even in case of sensible variations of the HCl eluate thus being particularly adequate for the routine application where strictly constant elution volumes cannot be anticipated;
To not negatively interfere with the ⁶⁸Ga complexation by the DOTA chelator. All these observations, lead to focus on formic acid for further development.

### 3.2 Final Formula and Detailed Composition

Based on all development performed on the formulation as above presented, the final chosen formulation of vial 2 is the following:

TABLE 26
Final formulation

| | Quality Standard | | |
|---|---|---|---|
| | (and Grade, if | | Quantity |
| Component | applicable) | Purpose | per vial |
| Formic acid | In-house | pH adjuster | 60 mg |
| Sodium hydroxide | In-house | pH adjuster | 56.5 mg |
| Water for injection | Ph. Eur/USP* | Solvent | qs |

1. A method for labeling a PSMA binding ligand with a radioactive isotope, preferably ⁶⁸Ga, ⁶⁷Ga or ⁶⁴Cu, said method comprising the steps of:
i. providing a first vial comprising said PSMA binding ligand, and optionally a bulking agent, in dried form,
ii. adding a solution of said radioactive isotope into said first vial, thereby obtaining a solution of said PSMA binding ligand with said radioactive isotope,
iii. mixing the solution obtained in ii. with at least a buffering agent and incubating it for a sufficient period of time for obtaining said PSMA binding ligand labeled with said radioactive isotope, and,
iv. optionally, adjusting the pH of the solution.

2. The method of claim 1, wherein said PSMA binding ligand is a compound of formula (I): 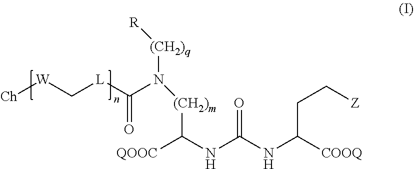
wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is -V-Y;
V is a bond or a C₁-C₆ alkylene, preferably V is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR′—C(NR″R′″)═NR″″, —S(O)R′, —S(O)₂R′, —S(O)₂NR′R″, —NRSO₂R′, —CN and —NO₂ in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR²—(C═O), —NR²—(C═S), —(C═O)—NR²—, and —(C═S)—NR²—, preferably, W is —(C═O)—NR²—;
each occurrence of L and W can be the same or different;
R² is H or C₁-C₄ alkyl, preferably R² is H;
n is an integer selected from the group consisting of 1, 2 and 3;
Ch is a chelating agent, typically DOTA.

3. The method of claim 2, wherein said PSMA binding ligand is a compound of formula (II):

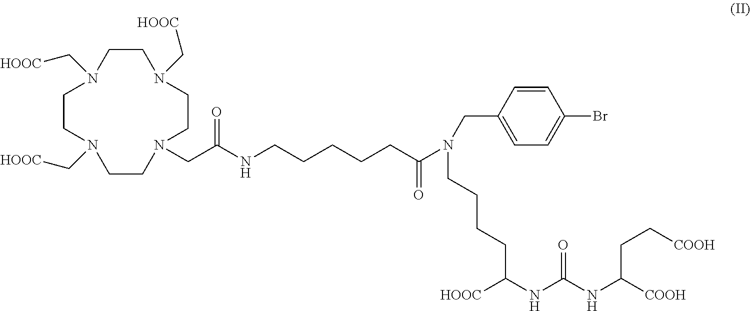 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

4. The method of claim 1, wherein said buffering agent comprises formic acid and sodium hydroxide as buffering agents.

5. A solution comprising a PSMA binding ligand labeled with a radioactive isotope, obtainable or obtained by the method of claim 1, for use as an injectable solution for in vivo detection of tumors, typically PSMA-expressing tumors, by imaging in a subject in need thereof.

6. A solution comprising a PSMA binding ligand of formula (II) labeled with ⁶⁸Ga, ⁶⁷Ga or ⁶⁴Cu obtainable or obtained by the method of claim 3, for use as an injectable solution for in vivo detection of tumors, typically PSMA-expressing tumors, by imaging in a subject in need thereof.

7. A powder for a solution for injection, comprising the following components in dried forms:
i. a PSMA binding ligand of formula (I): 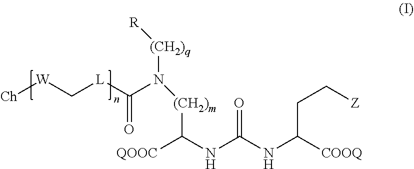
wherein:
Z is tetrazole or COOQ, preferably Z is COOQ;
Q is independently H or a protecting group, preferably Q is H;
m is an integer selected from the group consisting of 1, 2, 3, 4, and 5, preferably m is 4;
q is an integer selected from the group consisting of 1, 2, 3, 4, 5, and 6, preferably q is 1;
R is selected from the group consisting of C₆-C₁₀ aryl and heteroaryl containing 5 to 10 ring atoms, said aryl and heteroaryl being substituted 1 or more times with X;
X is -V-Y;
V is a bond or a C₁-C₆ alkylene, preferably V is a bond;
Y is a halogen;
L is a linker selected from the group consisting of C₁-C₆ alkylene, C₃-C₆ cycloalkylene and C₆-C₁₀ arylene, said alkylene, cycloalkylene and arylene being optionally substituted with one or more substituents selected from: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR′—C(NR″R″′)═NR″″, —S(O)R′, —S(O)₂R′, —S(O)₂NR′R″, —NRSO₂R′, —CN and —NO₂ in a number ranging from zero to 2m′, where m′ is the total number of carbon atoms in such groups. R′, R″, R′″ and R″″ each may independently refer to hydrogen, alkyl, heteroalkyl, cycloalkyl, heterocycloalkyl, aryl and heteroaryl;
W is selected from the group consisting of —NR²—(C═O), —NR²—(C═S), —(C═O)—NR²—, and —(C═S)—NR²—, preferably, W is —(C═O)—NR²—;
each occurrence of L and W can be the same or different;
R² is H or C₁-C₄ alkyl, preferably R² is H;
n is an integer selected from the group consisting of 1, 2 and 3;
Ch is a chelating agent, typically DOTA; and
ii. a bulking agent, preferably mannitol.

8. The powder for a solution for injection of claim 7, wherein said PSMA binding ligand is of formula (II):

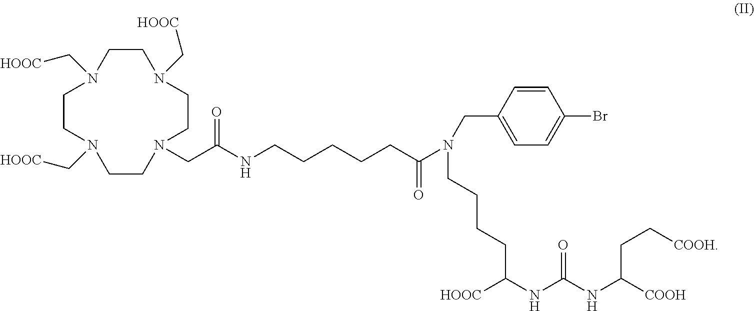 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

9. The powder for a solution for injection of claim 7, comprising the following components:
i. a PSMA binding ligand of formula (II) in an amount between 10 and 100 μg, preferably between 15 and 60 μg, even more preferably about 30 μg 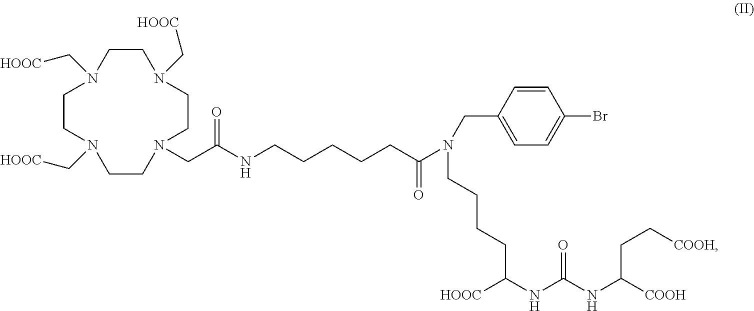 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]
and
ii. mannitol in an amount between 5 and 50 mg, preferably between 10 and 30 mg, even more preferably about 20 mg.

10. The powder for a solution for injection of claim 7, wherein said powder does not contain an antioxidant, for example the powder does not contain gentisic acid.

11. A kit for carrying out the method of claim 3, comprising
i. a first vial with the following components, preferably in dried forms:
i. a PSMA binding ligand of formula (II): 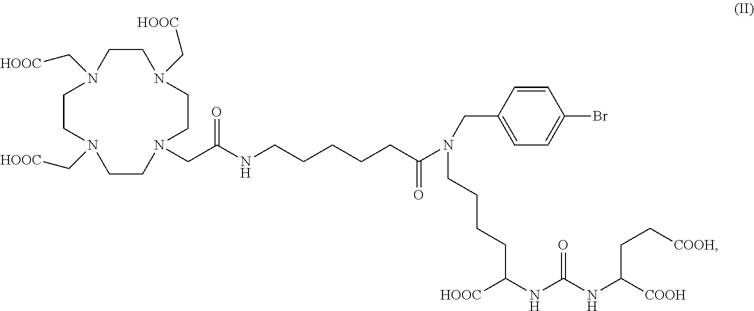 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]
and
ii. optionally a bulking agent, preferably mannitol, and
ii. a second vial comprising at least a buffering agent, preferably in dried form, and,
iii. optionally, an accessory cartridge for eluting a radioactive isotope generated by a radioactive isotope generator or a cyclotron.

12. The kit of claim 11, wherein the first vial comprises the following components:
i. a PSMA binding ligand of formula (II) in an amount between 10 and 100 μg, preferably between 15 and 60 μg, even more preferably about 30 μg; 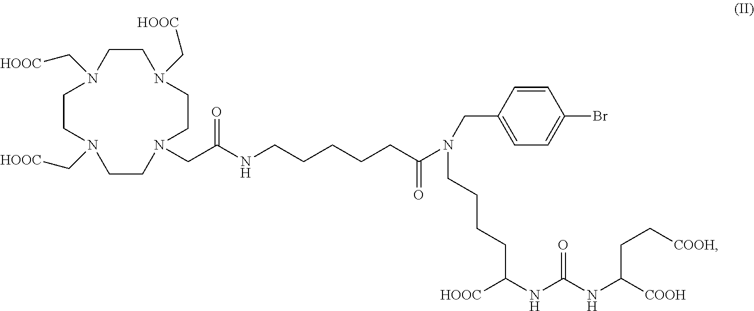 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]
and
ii. mannitol in an amount between 5 and 50 mg, preferably between 10 and 30 mg, even more preferably about 20 mg.

13. The kit of claim 11, wherein the second vial comprises buffering agents for maintaining a pH between 2.5 and 4.0, preferably between 2.8 and 4.0, more preferably between 3.0 and 4.0, and even more preferably between 3.2 and 3.8.

14. The kit of claim 11, wherein the kit does not contain an antioxidant, preferably the kit does not contain gentisic acid, and said second vial comprises buffering agents for maintaining a pH between 2.5 and 4.0, preferably between 2.8 and 4.0, more preferably between 3.0 and 4.0, and even more preferably between 3.2 and 3.8.

15. The kit of claim 11, wherein the second vial comprises formic acid and sodium hydroxide as buffering agents.

16. A kit for carrying out the method of claim 3, comprising
i. a single vial with the following components, preferably in dried forms:
i. a PSMA binding ligand of formula (II): 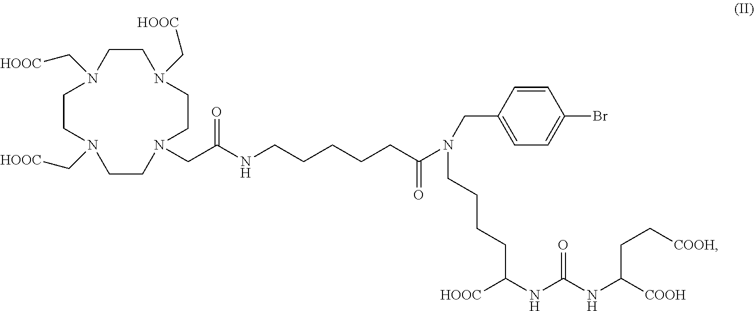 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]
and
ii. optionally a bulking agent, preferably mannitol, and
iii. at least a buffering agent, preferably in dried form, and,
ii. optionally, an accessory cartridge for eluting a radioactive isotope generated by a radioactive isotope generator or a cyclotron.

17. The kit of claim 16, wherein said single vial comprises the following components:
i. a PSMA binding ligand of formula (II) in an amount between 10 and 100 μg, preferably between 15 and 60 μg, even more preferably about 30 μg; 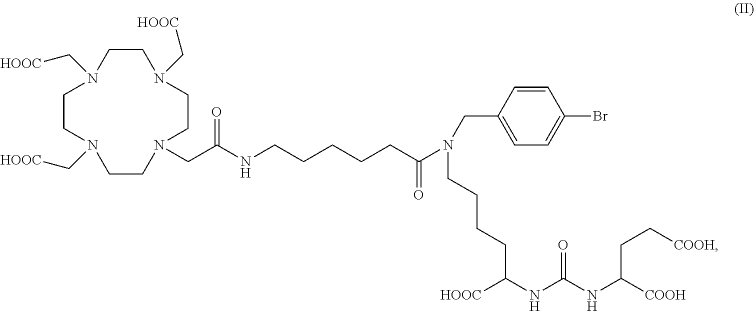 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]
and
ii. mannitol in an amount between 5 and 50 mg, preferably between 10 and 30 mg, even more preferably about 20 mg.

18. The kit of claim 16, wherein the single vial comprises buffering agents for maintaining a pH between 2.5 and 4.0, preferably between 2.8 and 4.0, more preferably between 3.0 and 4.0, and even more preferably between 3.2 and 3.8.

19. The kit of claim 16, wherein the kit does not contain an antioxidant, preferably the kit does not contain gentisic acid, and said second vial comprises buffering agents for maintaining a pH between 2.5 and 4.0, preferably between 2.8 and 4.0, more preferably between 3.0 and 4.0, and even more preferably between 3.2 and 3.8.

20. The kit of claim 16, wherein said single vial comprises formic acid and sodium hydroxide as buffering agents.
